(19) United States
(12) Patent Application Publication
(10) Pub. No.: US 2021/0062206 A1
(43) Pub. Date: Mar. 4, 2021

(54) Synthetic transcription factors

(71) Applicants: The Regents of the University of California, Oakland, CA (US); BASF Plant Science LP, Research Triangle Park, NC (US)

(72) Inventors: Patrick M. Shih, Berkeley, CA (US); Fernando Ferreira, Cary, NC (US); Marianela Rodriguez-Carres, Durham, NC (US)

(21) Appl. No.: 16/923,931

(22) Filed: Jul. 8, 2020

Related U.S. Application Data
(60) Provisional application No. 62/871,611, filed on Jul. 8, 2019.

(51) Int. Cl. C12N 15/82; C07K 14/395

(52) U.S. Cl. CPC C12N 15/8241; C07K 2319/09; C07K 2319/80; C07K 14/395

(57) ABSTRACT

The present invention provides for a synthetic transcription factor (TF) comprising (a) a DNA-binding domain of a transcription factor linked to (b) an activator domain or repressor domain, and (c) a nuclear localization sequence (NLS).

### CROSS REFERENCE TO RELATED APPLICATIONS

This application claims priority to U.S. Provisional Patent Application Ser. No. 62/871,611, filed Jul. 8, 2019, which is incorporated by reference in its entirety.

### STATEMENT OF GOVERNMENTAL SUPPORT

The invention was made with government support under Contract Nos. DE-ACO2-05CH11231 awarded by the U.S. Department of Energy. The government has certain rights in the invention.

### FIELD OF THE INVENTION

The present invention is in the field of regulating gene expression in plants.

### BACKGROUND OF THE INVENTION

Plants offer a unique platform to address many imminent challenges that face society, as future engineering efforts hold promise in promoting sustainable agriculture, renewable energy, and green technologies.. However, the tools to effectively modify and engineer plants are still in their infancy. One major hurdle has been the development of tools and genetic parts that enable precise control of transgene expression in plants.

Many genetic and metabolic engineering efforts require the robust and accurate control of genes to optimize desired pathways, increase flux, and avoid bottlenecks^{4,5}. The ability to modulate gene expression provides an efficient and simple means to address these tasks. However, the vast majority of plant engineering efforts have been limited to utilizing a small number of characterized constitutive promoters, which may result in unintended pleiotropic effects or toxicity issues as well as limited expression strength. We generated a diverse library of orthogonal synthetic promoter-transcription factor pairs and characterized them using a high throughput transient expression assay in Nicotiana benthamiana. This library of synthetic transcriptional regulators was designed to modulate transcription with both cis and trans elements and introduces a novel method for the design and construction of new promoter/transcription factor pairs for plant synthetic biology.

### SUMMARY OF THE INVENTION

The present invention provides for a synthetic transcription factor (TF) comprising (a) a DNA-binding domain of a transcription factor linked to (b) an activator domain or repressor domain, and (c) a nuclear localization sequence (NLS).

In some embodiments, the DNA-binding domain is a DNA-binding domain of a eukaryotic TF or a prokaryotic TF. In some embodiments, the DNA-binding domain is a DNA-binding domain of a eukaryotic TF.

In some embodiments, the eukaryotic TF is a yeast TF. In some embodiments, the yeast TF is a Saccharomyces TF. In some embodiments, the Saccharomyces TF is a Saccharomyces cerevisiae TF.

In some embodiments, the S. cerevisiae TF is Gal4, YAP1, GAT1, MATAL1, MATAL2, MCM1, Abf1, Adr1, Ash1, Gcn4, Gcr1, Hap4, Hsf1, Ime1, Ino2/Ino4, Leu3, Lys14, Matα2, Mga2, Met4, Mig1, Rap1, Rgt1, Rlm1, Smp1, Rme1, Rox1, Rtg3, Spt23, Tea1, Ume6, or Zap1. In some embodiments, the S. cerevisiae TF is Gal4, YAP1, GAT1, MATAL1, MATAL2, or MCM1.

In some embodiments, the S. cerevisiae TF is Gal4. In some embodiments, the DNA-binding domain comprises the amino acid sequence of Gal4 or MKLLSSIEQA CDICRLKKLK CSKEKPKCAK CLKNNWECRY SPKTKRSPLT RAHLTEVESR LERLEQLFLL IFPREDLDMI LKMDSLQDIK ALLTGLFVQD NVNKDAVTDR LASVETDMPL TLRQHRISAT SSSEESSNKG QRQLTV (SEQ ID NO:32).

In some embodiments, the S. cerevisiae TF is YAP1. In some embodiments, the DNA-binding domain comprises the amino acid sequence of YAP 1, PETKQKR TAQNRAAQRA FRERKERKMK ELEKKVQSLE SIQQQNEVEA TFLRDQLITL VNELKKY (SEQ ID NO:33) or KQ DLDPETKQKR TAQNRAAQRA FRERKERKMK ELEKKVQSLE SIQQQNEVEA TFLRDQLITL VNELKKYRPE TRNDSKVLEY LARRDPNL (SEQ ID NO:34).

In some embodiments, the S. cerevisiae TF is GAT1. In some embodiments, the DNA-binding domain comprises the amino acid sequence of GAT1, IFTNNLP FLNNNSINNN HSHNSSHNNN SPSIANNTNA NTNTNTSAST NTNSPLL (SEQ ID NO:35) or D DHFIFTNNLP FLNNNSINNN HSHNSSHNNN SPSIANNTNA NTNTNTSAST NTNSPLLRRN PSP (SEQ ID NO:36).

In some embodiments, the S. cerevisiae TF is MATAL1. In some embodiments, the DNA-binding domain comprises the amino acid sequence of MATAL1 or KKEKS PKGKSSISPQ ARAFLEQVFR RKQSLNSKEK EEVAKKCGIT PLQVRVWFIN KRMRSK (SEQ ID NO:37).

In some embodiments, the S. cerevisiae TF is MATAL2. In some embodiments, the DNA-binding domain comprises the amino acid sequence of MATAL2 or STKP YRGHRFTKEN VRILESWFAK NIENPYLDTK GLENLMKNTS LSRIQIKNWV SNRRRKEKTI TIAP (SEQ ID NO:38).

In some embodiments, the S. cerevisiae TF is MCM1. In some embodiments, the DNA-binding domain comprises the amino acid sequence of MCM1, RRK IEIKFIENKT RRHVTFSKRK HGIMKKAFEL SVLTGTQVLL LVVSETGLVY TF (SEQ ID NO:39) or KERRK IEIKFIENKT RRHVTFSKRK HGIMKKAFEL SVLTGTQVLL LVVSETGLVY TFSTPKFEPI VTQQEGRNLI QACLNA (SEQ ID NO:40).

In some embodiments, the S. cerevisiae TF is Rap1. In some embodiments, the DNA-binding domain comprises the amino acid sequence of Rap1, or GXXIRXRF (wherein X is any amino acid) (SEQ ID NO:41), G(G, P, A or R)(S or A)IRXRF (wherein X is any amino acid) (SEQ ID NO:42), or GNSIRHRFRV(SEQ ID NO:43).

In some embodiments, the synthetic TF comprises the activator domain which is a herpes simplex virus VP16, maize C1, or a yeast activator domain.

In some embodiments, the activator domain is the herpes simplex virus VP16 activator domain comprising the amino acid sequence: APPTDVSLGD ELHLDGEDVA MAHADALDDF DLDMLGDGDS PGPGFTPHDS APYGALDMAD FEFEQMFTDA LGIDEYGG (SEQ ID NO:44).

In some embodiments, the activator domain is the maize C1 activator domain comprising the amino acid sequence: CETGQNSAAH RADPDSAGTT TTSAAAVWAP KAVRCTGGLF FFHRDTTPAH AGETATPMAG GGGGGGGEAG SSDDCSSAAS VSLRVGSHDE PCFSGDGDGD WMDDVRALAS FLESDEDWLR CQTA (SEQ ID NO:45).

In some embodiments, the activator domain is the yeast activator domain. In some embodiments, the yeast activator domain is a Saccharomyces activator domain. In some embodiments, the Saccharomyces activator domain is a Saccharomyces cerevisiae activator domain. In some embodiments, the S. cerevisiae activator domain is a Gal4, YAP1, GAT1, MATAL1, MATAL2, MCM1, Abf1, Adr1, Ash1, Gcn4, Gcr1, Hap4, Hsf1, Ime1, Ino2/Ino4, Leu3, Lys14, Mga2, Met4, Rap1, Rlm1, Smp1, Rtg3, Spt23, Teal, Ume6, or Zap1 activator domain.

The synthetic TF of claim 29, wherein the S. cerevisiae activator domain is a Rap1 activator domain. In some embodiments, the DNA-binding domain comprises the amino acid sequence of a Rap1, or IPEXELLDEX TXNFXSXLXX DLSXXXNXXX FEYXXEIAE (wherein X is any amino acid) (SEQ ID NO:46), (S or Q) (Y or F) X IPE (N or G) ELLDE (D or E) T(M or L) NF (I or L) SXL (K or R) (N or R) DLSX (I or L) (S or E) NXX (P or A) FEYXXEIAE (wherein X is any amino acid) (SEQ ID NO:47), SYA IPENELLDED TMNFISSLKN DLSNISNSLP FEYPHEIAE, SYP IPENELLDEE TMNFISNLKN DLSKLENNLP FEYSPEIAE, QFS IPEGELLDEE TLNFLSGLRR DLSRIENGMA FEYPQEIAE (SEQ ID NO:48).

In some embodiments, the synthetic TF comprises the repressor domain. In some embodiments, the repressor domain comprises an EAR motif, TLLLFR motif, R/KLFGV motif, LxLxPP motif, or a yeast repressor domain.

In some embodiments, the repressor domain comprises the EAR motif. In some embodiments, the repressor domain comprises the amino acid sequence LDLDLELRLGFA (SEQ ID NO:49) (SRDX), IDLDLNLAPPME (SEQ ID NO:50), QDLDLELRLGFA (SEQ ID NO:51), LQLGL, LDL (N or E) L (SEQ ID NO:52), (L or F) DLN (L or F) XP (wherein X is any amino acid) (SEQ ID NO:53), LXLXL (wherein X is any amino acid) (SEQ ID NO:54), or DLNXXP (wherein X is any amino acid) (SEQ ID NO:55).

In some embodiments, the repressor domain comprises the TLLLFR motif.

In some embodiments, the repressor domain comprises the R/KLFGV motif. In some embodiments, the repressor domain comprises the amino acid sequence (R or K) LFGV (SEQ ID NO:56), (L or V) (R or K) LFGVX (M, V or L) (SEQ ID NO:57), GNSKTL RLFGV NMEC (SEQ ID NO:58), GSSRTV RLFGV NLEC (SEQ ID NO: 59), GSSRTV RLFGV NLEC (SEQ ID NO:60), GSSRTV RLFGV NLEC (SEQ ID NO:61), TAGKRL RLFGV NMEC (SEQ ID NO:62), RGEKRL RLFGV DMEC (SEQ ID NO:63), STTKKL RLFGV DVEE (SEQ ID NO:64), DAGRVL RLFGV NISP (SEQ ID NO:65), PVQVVV RLFGV DIFN (SEQ ID NO:66), ETGRVM RLFGV DISL (SEQ ID NO:67), PVQTVV RLFGV NIFN (SEQ ID NO:68), KAVTNF RLFGV SLAI (SEQ ID NO:69), KTGTNF RLFGV TLDT (SEQ ID NO:70), KTGTNF RLFGV SLVT (SEQ ID NO:71), KAGTNF RLFGV TLDT (SEQ ID NO:72), KAGTNF RLFGV SLAT (SEQ ID NO:73), NAVASF RLFGV SLAT (SEQ ID NO:74), GVGEGL KLFGV WLKG (SEQ ID NO:75), EEEASP RLFGV PIGL (SEQ ID NO:76), GEDLTP RLFGV SIGV (SEQ ID NO:77), EEDEGL KLFGV KLE (SEQ ID NO:78), SNMRKT KLFGV SLPS (SEQ ID NO:79), GSSSAV KLFGV RLTD (SEQ ID NO:80), CPNRGV KLFGV RLTE (SEQ ID NO:81), YQTRVV RLFGV HLDT (SEQ ID NO:82), VNKASV KLFGV NISS (SEQ ID NO:83), ASLTKG KLFGV DLM (SEQ ID NO:84), QSLSKA RLFGV DLN (SEQ ID NO:85), or RLAAYA KLFGV PFE (SEQ ID NO:86).

In some embodiments, the repressor domain comprises the LxLxPP motif.

In some embodiments, the repressor domain is the yeast repressor domain. In some embodiments, the yeast repressor domain is a Saccharomyces repressor domain. In some embodiments, the Saccharomyces repressor domain is a Saccharomyces cerevisiae repressor domain. In some embodiments, the S. cerevisiae repressor domain is an Ash1, Matα2, Mig1, Rap1, Rgt1, Rme1, Rox1, or Ume6 repressor domain.

In some embodiments, the NLS is monopartite. In some embodiments, the NLS comprises the amino acid sequence K-K/R-X-K/R (SEQ ID NO:87), PKKKRKV (SV40 Large T-antigen) (SEQ ID NO:88), PAAKRVKLD (c-Myc) (SEQ ID NO:89) or KLKIKRPVK (TUS-protein) (SEQ ID NO:90).

In some embodiments, the NLS is bipartite. In some embodiments, the NLS comprises the amino acid sequence KRX₁₀KKKK (SEQ ID NO:91), KRPAATKKAGQAKKKK (SEQ ID NO:92) or AVKRPAATKKAGQAKKKKLD (nucleoplasmin NLS) (SEQ ID NO:93) or MSRRRKANPTKLSENAKKLAKEVEN (EGL-13) (SEQ ID NO:94).

In some embodiments, the NLS comprises a M9 domain or PY-NLS motif. In some embodiments, the NLS comprises the M9 domain comprising the amino acid sequence (a) one or more of YNDFGNYN (SEQ ID NO:95) or FGNYN (SEQ ID NO:96), SN-F/Y-GPMK (SEQ ID NO:97), N-F/Y-GG (SEQ ID NO:98), GPYGGG (SEQ ID NO:99), (b) GNYNNQS SNFGPMKGGN FGGRSSGPYG GGGQYFAKPR NQGGY (hnRNP A1) (SEQ ID NO:100), (c) FGNYNQQPSN YGPMKSGNFG GSRNMGGPYG GGNYGPGGSG GSGGY(hnRNP A2/B1) (SEQ ID NO:101), (d) FGNYNSQSSS NFGPMKGGNY GGRNSGPYGG GYGGGSASSS SGY (Xenopus RNP A1) (SEQ ID NO:102), or (e) FGNYNQQSSN YGPMKSGGNF GGNRSMGGGP YGGGNYGPGN ASGGNGGGY (Xenopus RNP A2) (SEQ ID NO:103).

In some embodiments, the NLS comprises the amino acid sequence KIPIK (yeast Matα2) (SEQ ID NO:104).

In some embodiments, wherein any two, or all, of the DNA-binding domain, the activator domain, the repressor domain, and the NLS are heterologous to each other.

In some embodiments, wherein one or more, or all, of the DNA-binding domain, the activator domain, the repressor domain, and the NLS are obtained or derived from a non-viral organism.

In some embodiments, the DNA-binding domain, the NLS, and the activator domain or repressor domain are linked in this order from N- to C-terminus. Exemplary synthetic TF include, but are not limited to, the following:

In some embodiments, the synthetic TF comprises the indicated domains linked in the following order from N- to C-terminus: MCM1-SV40-VP16. In one particular empbodiment, the synthetic TF comprises the following amino acid sequence:

(SEQ ID NO: 1)
MSDIEEGTPTNNGQQKERRKIEIKFIENKTRRHVTFSKRKHGIMKKAFEL
SVLTGTQVLLLVVSETGLVYTFSTPKFEPIVTQQEGRNLIQACLNAPDDE
EEDEEEDGDDDDDDDDDGNDMQRQQPQQQQPQQQQQVLNAHANSLGHLNQ
DQVPAGALKQEVKSQLLGGANPNQNSMIQQQQHHTQNSQPQQQQQQQPQQ
QMSQQQMSQHPRPQQGIPHPQQSQPQQQQQQQQQLQQQQQQQQQQPLTGI
HQPHQQAFANAASPYLNAEQNAAYQQYFQEPQQGQYGPKKKRKVAPPTDV
SLGDELHLDGEDVAMAHADALDDFDLDMLGDGDSPGPGFTPHDSAPYGAL
DMADFEFEQMFTDALGIDEYGG (MCM1 [1-286]; SV40
[288-294]; VP16 [295-372]).

In some embodiments, the synthetic TF comprises the indicated domains linked in the following order from N- to C-terminus: MCM1-DBDa-SV40-VP16. In one particular empbodiment, the synthetic TF comprises the following amino acid sequence:

(SEQ ID NO: 2)
MRRKIEIKFIENKTRRHVTFSKRKHGIMKKAFELSVLTGTQVLLL
VVSETGLVYTFGPKKKRKVAPPTDVSLGDELHLDGEDVAMAHADA
LDDFDLDMLGDGDSPGPGFTPHDSAPYGALDMADFEFEQMFTDAL
GIDEYGG
(DBDa [2-56], (18-72 of native seq);
SV40 [58-64]; VP16 [65-142]).

In some embodiments, the synthetic TF comprises the indicated domains linked in the following order from N- to C-terminus: MCM1-DBDb-SV40-VP16. In one particular empbodiment, the synthetic TF comprises the following amino acid sequence:

(SEQ ID NO: 3)
MKERRKIEIKFIENKTRRHVTFSKRKHGIMKKAFELSVLTGTQVL
LLVVSETGLVYTFSTPKFEPIVTQQEGRNLIQACLNAGPKKKRKV
APPTDVSLGDELHLDGEDVAMAHADALDDFDLDMLGDGDSPGPGF
TPHDSAPYGALDMADFEFEQMFTDALGIDEYGG
(DBDb [2-82], (16-96 of native seq);
SV40 [84-90]; VP16 [91-168]).

In some embodiments, the synthetic TF comprises the indicated domains linked in the following order from N- to C-terminus: MCM1-SV40-C1. In one particular empbodiment, the synthetic TF comprises the following amino acid sequence:

(SEQ ID NO: 4)
MSDIEEGTPTNNGQQKERRKIEIKFIENKTRRHVTFSKRKHGIMKK
AFELSVLTGTQVLLLVVSETGLVYTFSTPKFEPIVTQQEGRNLIQA
CLNAPDDEEEDEEEDGDDDDDDDDDGNDMQRQQPQQQQPQQQQQVL
NAHANSLGHLNQDQVPAGALKQEVKSQLLGGANPNQNSMIQQQQHH
TQNSQPQQQQQQQPQQQMSQQQMSQHPRPQQGIPHPQQSQPQQQQQ
QQQQLQQQQQQQQQQPLTGIHQPHQQAFANAASPYLNAEQNAAYQQ
YFQEPQQGQYGPKKKRKVCETGQNSAAHRADPDSAGTTTTSAAAVW
APKAVRCTGGLFFFHRDTTPAHAGETATPMAGGGGGGGGEAGSSDD
CSSAASVSLRVGSHDEPCFSGDGDGDWMDDVRALASFLESDEDWLR
CQTA
(MCM1 [1-286]; SV40 [288-294]; C1 [295-418]).

In some embodiments, the synthetic TF comprises the indicated domains linked in the following order from N- to C-terminus: MCM1-DBDa-SV40-C1. In one particular empbodiment, the synthetic TF comprises the following amino acid sequence:

(SEQ ID NO: 5)
MRRKIEIKFIENKTRRHVTFSKRKHGIMKKAFELSVLTGTQVLLL
VVSETGLVYTFGPKKKRKVCETGQNSAAHRADPDSAGTTTTSAAA
VWAPKAVRCTGGLFFFHRDTTPAHAGETATPMAGGGGGGGGEAGS
SDDCSSAASVSLRVGSHDEPCFSGDGDGDWMDDVRALASFLESDE
DWLRCQTA
(DBDa [2-56], (18-72 of native seq);
SV40 [58-64]; C1 [65-188]).

In some embodiments, the synthetic TF comprises the indicated domains linked in the following order from N- to C-terminus: MCM1-DBDb-SV40-C1. In one particular empbodiment, the synthetic TF comprises the following amino acid sequence:

(SEQ ID NO: 6)
MKERRKIEIKFIENKTRRHVTFSKRKHGIMKKAFELSVLTGTQVLL
LVVSETGLVYTFSTPKFEPIVTQQEGRNLIQACLNAGPKKKRKVCE
TGQNSAAHRADPDSAGTTTTSAAAVWAPKAVRCTGGLFFFHRDTTP
AHAGETATPMAGGGGGGGGEAGSSDDCSSAASVSLRVGSHDEPCFS
GDGDGDWMDDVRALASFLESDEDWLRCQTA
(DBDb [2-82], (16-96 of native
seq); SV40 [84-90]; C1 [91-214]).

In some embodiments, the synthetic TF comprises the indicated domains linked in the following order from N- to C-terminus: MATAL1-SV40-VP16. In one particular empbodiment, the synthetic TF comprises the following amino acid sequence:

(SEQ ID NO: 7)
MDDICSMAENINRTLFNILGTEIDEINLNTNNLYNFIMESNLTKVE
QHTLHKNISNNRLEIYHHIKKEKSPKGKSSISPQARAFLEQVFRRK
QSLNSKEKEEVAKKCGITPLQVRVWFINKRMRSKGPKKKRKVAPPT
DVSLGDELHLDGEDVAMAHADALDDFDLDMLGDGDSPGPGFTPHDS
APYGALDMADFEFEQMFTDALGIDEYGG
(MATAL1 [1-126]; SV40 [128-134];
VP16 [135-212]).

In some embodiments, the synthetic TF comprises the indicated domains linked in the following order from N- to C-terminus: MATAL1-DBD-SV40-VP16. In one particular empbodiment, the synthetic TF comprises the following amino acid sequence:

(SEQ ID NO: 8)
MKKEKSPKGKSSISPQARAFLEQVFRRKQSLNSKEKEEVAKKCGIT
PLQVRVWFINKRMRSKGPKKKRKVAPPTDVSLGDELHLDGEDVAMA
HADALDDFDLDMLGDGDSPGPGFTPHDSAPYGALDMADFEFEQMFT
DALGIDEYGG
(DBD [2-62], (66-126 of native seq);
SV40 [64-70]; VP16 [71-148]).

In some embodiments, the synthetic TF comprises the indicated domains linked in the following order from N- to C-terminus: MATAL1-SV40-C1. In one particular empbodiment, the synthetic TF comprises the following amino acid sequence:

(SEQ ID NO: 9)
MDDICSMAENINRTLFNILGTEIDEINLNTNNLYNFIMESNLTKV
EQHTLHKNISNNRLEIYHHIKKEKSPKGKSSISPQARAFLEQVFR
RKQSLNSKEKEEVAKKCGITPLQVRVWFINKRMRSKGPKKKRKVC
ETGQNSAAHRADPDSAGTTTTSAAAVWAPKAVRCTGGLFFFHRDT
TPAHAGETATPMAGGGGGGGGEAGSSDDCSSAASVSLRVGSHDEP
CFSGDGDGDWMDDVRALASFLESDEDWLRCQTA
(MATAL1 [1-126]; SV40 [128-134];
C1 [135-258]).

In some embodiments, the synthetic TF comprises the indicated domains linked in the following order from N- to C-terminus: MATAL1-DBD-SV40-C1. In one particular empbodiment, the synthetic TF comprises the following amino acid sequence:

(SEQ ID NO: 10)
MKKEKSPKGKSSISPQARAFLEQVFRRKQSLNSKEKEEVAKKCGI
TPLQVRVWFINKRMRSKGPKKKRKVCETGQNSAAHRADPDSAGTT
TTSAAAVWAPKAVRCTGGLFFFHRDTTPAHAGETATPMAGGGGGG
GGEAGSSDDCSSAASVSLRVGSHDEPCFSGDGDGDWMDDVRALAS
FLESDEDWLRCQTA
(DBD [2-62], (66-126 of native seq);
SV40 [64-70]; C1 [71-194]).

In some embodiments, the synthetic TF comprises the indicated domains linked in the following order from N- to C-terminus: MATAL2-SV40-VP16. In one particular empbodiment, the synthetic TF comprises the following amino acid sequence:

(SEQ ID NO: 11)
MNKIPIKDLLNPQITDEFKSSILDINKKLFSICCNLPKLPESVTT
EEEVELRDILGFLSRANKNRKISDEEKKLLQTTSQLTTTITVLLK
EMRSIENDRSNYQLTQKNKSADGLVFNVVTQDMINKSTKPYRGHR
FTKENVRILESWFAKNIENPYLDTKGLENLMKNTSLSRIQIKNWV
SNRRRKEKTITIAPELADLLSGEPLAKKKEGPKKKRKVAPPTDVS
LGDELHLDGEDVAMAHADALDDFDLDMLGDGDSPGPGFTPHDSAP
YGALDMADFEFEQMFTDALGIDEYGG
(MATAL2 [1-210]; SV40 [212-218];
VP16 [219-296]).

In some embodiments, the synthetic TF comprises the indicated domains linked in the following order from N- to C-terminus: MATAL2-DBD-SV40-VP16. In one particular empbodiment, the synthetic TF comprises the following amino acid sequence:

(SEQ ID NO: 12)
MSTKPYRGHRFTKENVRILESWFAKNIENPYLDTKGLENLMKNTS
LSRIQIKNWVSNRRRKEKTITIAPGPKKKRKVAPPTDVSLGDELH
LDGEDVAMAHADALDDFDLDMLGDGDSPGPGFTPHDSAPYGALDM
ADFEFEQMFTDALGIDEYGG
(DBD [2-69], (127-194 of native
seq); SV40 [71-77]; VP16 [78-155]).

In some embodiments, the synthetic TF comprises the indicated domains linked in the following order from N- to C-terminus: MATAL2-SV40-C1. In one particular empbodiment, the synthetic TF comprises the following amino acid sequence:

(SEQ ID NO: 13)
MNKIPIKDLLNPQITDEFKSSILDINKKLFSICCNLPKLPESVTT
EEEVELRDILGFLSRANKNRKISDEEKKLLQTTSQLTTTITVLLK
EMRSIENDRSNYQLTQKNKSADGLVFNVVTQDMINKSTKPYRGHR
FTKENVRILESWFAKNIENPYLDTKGLENLMKNTSLSRIQIKNWV
SNRRRKEKTITIAPELADLLSGEPLAKKKEGPKKKRKVCETGQNS
AAHRADPDSAGTTTTSAAAVWAPKAVRCTGGLFFFHRDTTPAHAG
ETATPMAGGGGGGGGEAGSSDDCSSAASVSLRVGSHDEPCFSGDG
DGDWMDDVRALASFLESDEDWLRCQTA
(MATAL2 [1-210]; SV40 [212-218];
C1 [219-342]).

In some embodiments, the synthetic TF comprises the indicated domains linked in the following order from N- to C-terminus: MATAL2-DBD-SV40-C1. In one particular empbodiment, the synthetic TF comprises the following amino acid sequence:

(SEQ ID NO: 14)
MSTKPYRGHRFTKENVRILESWFAKNIENPYLDTKGLENLMKNTS
LSRIQIKNWVSNRRRKEKTITIAPGPKKKRKVCETGQNSAAHRAD
PDSAGTTTTSAAAVWAPKAVRCTGGLFFFHRDTTPAHAGETATPM
AGGGGGGGGEAGSSDDCSSAASVSLRVGSHDEPCFSGDGDGDWMD
DVRALASFLESDEDWLRCQTA
(DBD [2-69], (127-194 of native seq);
SV40 [71-77]; C1 [78-201]).

In some embodiments, the synthetic TF comprises the indicated domains linked in the following order from N- to C-terminus: Yap1-SV40-VP16. In one particular empbodiment, the synthetic TF comprises the following amino acid sequence:

(SEQ ID NO: 15)
MSVSTAKRSLDVVSPGSLAEFEGSKSRHDEIENEHRRTGTRDGED
SEQPKKKGSKTSKKQDLDPETKQKRTAQNRAAQRAFRERKERKMK
ELEKKVQSLESIQQQNEVEATFLRDQLITLVNELKKYRPETRNDS
KVLEYLARRDPNLHFSKNNVNHSNSEPIDTPNDDIQENVKQKMNF
TFQYPLDNDNDNDNSKNVGKQLPSPNDPSHSAPMPINQTQKKLSD
ATDSSSATLDSLSNSNDVLNNTPNSSTSMDWLDNVIYTNRFVSGD
DGSNSKTKNLDSNMFSNDFNFENQFDEQVSEFCSKMNQVCGTRQC
PIPKKPISALDKEVFASSSILSSNSPALTNTWESHSNITDNTPAN
VIATDATKYENSFSGFGRLGFDMSANHYVVNDNSTGSTDSTGSTG
NKNKKNNNNSDDVLPFISESPFDMNQVTNFFSPGSTGIGNNAASN
TNPSLLQSSKEDIPFINANLAFPDDNSTNIQLQPFSESQSQNKFD
YDMFFRDSSKEGNNLFGEFLEDDDDDKKAANMSDDESSLIKNQLI
NEEPELPKQYLQSVPGNESEISQKNGSSLQNADKINNGNDNDNDN
DVVPSKEGSLLRCSEIWDRITTHPKYSDIDVDGLCSELMAKAKCS
ERGVVINAEDVQLALNKHMNGPKKKRKVAPPTDVSLGDELHLDGE
DVAMAHADALDDFDLDMLGDGDSPGPGFTPHDSAPYGALDMADFE
FEQMFTDALGIDEYGG
(Yap1 [1-650]; SV40 [652-658];
VP16 [659-736]).

In some embodiments, the synthetic TF comprises the indicated domains linked in the following order from N- to C-terminus: Yap1-TF1a-SV40-VP16. In one particular empbodiment, the synthetic TF comprises the following amino acid sequence:

(SEQ ID NO: 16)
MPETKQKRTAQNRAAQRAFRERKERKMKELEKKVQSLESIQQQNE
VEATFLRDQLITLVNELKKYGPKKKRKVAPPTDVSLGDELHLDGE
DVAMAHADALDDFDLDMLGDGDSPGPGFTPHDSAPYGALDMADFE
FEQMFTDALGIDEYGG
(TF1a [2-65],(64-127 of native
seq); SV40 [67-73]; VP16 [74-151]).

In some embodiments, the synthetic TF comprises the indicated domains linked in the following order from N- to C-terminus: Yap1-TF1b-SV40-VP16. In one particular empbodiment, the synthetic TF comprises the following amino acid sequence:

(SEQ ID NO: 17)
MKQDLDPETKQKRTAQNRAAQRAFRERKERKMKELEKKVQSLESIQQQNE
VEATFLRDQLITLVNELKKYRPETRNDSKVLEYLARRDPNLGPKKKRKVA
PPTDVSLGDELHLDGEDVAMAHADALDDFDLDMLGDGDSPGPGFTPHDSA
PYGALDMADFEFEQMFTDALGIDEYGG
(TF1b [2-91], (59-148 of native seq);
SV40 [93-99]; VP16 [100-177]).

In some embodiments, the synthetic TF comprises the indicated domains linked in the following order from N- to C-terminus: Yap1-SV40-C1. In one particular empbodiment, the synthetic TF comprises the following amino acid sequence:

(SEQ ID NO: 18)
MSVSTAKRSLDVVSPGSLAEFEGSKSRHDEIENEHRRTGTRDGEDSEQPK
KKGSKTSKKQDLDPETKQKRTAQNRAAQRAFRERKERKMKELEKKVQSLE
SIQQQNEVEATFLRDQLITLVNELKKYRPETRNDSKVLEYLARRDPNLHF
SKNNVNHSNSEPIDTPNDDIQENVKQKMNFTFQYPLDNDNDNDNSKNVGK
QLPSPNDPSHSAPMPINQTQKKLSDATDSSSATLDSLSNSNDVLNNTPNS
STSMDWLDNVIYTNRFVSGDDGSNSKTKNLDSNMFSNDFNFENQFDEQVS
EFCSKMNQVCGTRQCPIPKKPISALDKEVFASSSILSSNSPALTNTWESH
SNITDNTPANVIATDATKYENSFSGFGRLGFDMSANHYVVNDNSTGSTDS
TGSTGNKNKKNNNNSDDVLPFISESPFDMNQVTNFFSPGSTGIGNNAASN
TNPSLLQSSKEDIPFINANLAFPDDNSTNIQLQPFSESQSQNKFDYDMFF
RDSSKEGNNLFGEFLEDDDDDKKAANMSDDESSLIKNQLINEEPELPKQY
LQSVPGNESEISQKNGSSLQNADKINNGNDNDNDNDVVPSKEGSLLRCSE
IWDRITTHPKYSDIDVDGLCSELMAKAKCSERGVVINAEDVQLALNKHMN
GPKKKRKVCETGQNSAAHRADPDSAGTTTTSAAAVWAPKAVRCTGGLFFF
HRDTTPAHAGETATPMAGGGGGGGGEAGSSDDCSSAASVSLRVGSHDEPC
FSGDGDGDWMDDVRALASFLESDEDWLRCQTA
(Yap1 [1-650]; SV40 [652-658]; C1 [659-782]).

In some embodiments, the synthetic TF comprises the indicated domains linked in the following order from N- to C-terminus: Yap1-TF1a-SV40-C1. In one particular empbodiment, the synthetic TF comprises the following amino acid sequence:

(SEQ ID NO: 19)
MPETKQKRTAQNRAAQRAFRERKERKMKELEKKVQSLESIQQQNEVEATF
LRDQLITLVNELKKYGPKKKRKVCETGQNSAAHRADPDSAGTTTTSAAAV
WAPKAVRCTGGLFFFHRDTTPAHAGETATPMAGGGGGGGGEAGSSDDCSS
AASVSLRVGSHDEPCFSGDGDGDWMDDVRALASFLESDEDWLRCQTA
(TF1a [2-65], (64-127 of native seq);
SV40 [67-73]; C1 [74-197]).

In some embodiments, the synthetic TF comprises the indicated domains linked in the following order from N- to C-terminus: Yap1-TF1b-SV40-C1. In one particular empbodiment, the synthetic TF comprises the following amino acid sequence:

(SEQ ID NO: 20)
MKQDLDPETKOKRTAQNRAAQRAFRERKERKMKELEKKVQSLESIQQQNE
VEATFLRDQLITLVNELKKYRPETRNDSKVLEYLARRDPNLGPKKKRKVC
ETGQNSAAHRADPDSAGTTTTSAAAVWAPKAVRCTGGLFFFHRDTTPAHA
GETATPMAGGGGGGGGEAGSSDDCSSAASVSLRVGSHDEPCFSGDGDGDW
MDDVRALASFLESDEDWLRCQTA
(TF1b [2-91], (59-148 of native seq);
SV40 [93-99]; C1 [100-223]).

In some embodiments, the synthetic TF comprises the indicated domains linked in the following order from N- to C-terminus: Gat1-SV40-VP16. In one particular empbodiment, the synthetic TF comprises the following amino acid sequence:

(SEQ ID NO: 21)
MHVFFPLLFRPSPVLFIACAYIYIDIYIHCTRCTVVNITMSTNRVPNLDP
DLNLNKEIWDLYSSAQKILPDSNRILNLSWRLHNRTSFHRINRIMQHSNS
IMDFSASPFASGVNAAGPGNNDLDDTDTDNQQFFLSDMNLNGSSVFENVF
DDDDDDDDVETHSIVHSDLLNDMDSASQRASHNASGFPNFLDTSCSSSFD
DHFIFTNNLPFLNNNSINNNHSHNSSHNNNSPSIANNTNANTNTNTSAST
NTNSPLLRRNPSPSIVKPGSRRNSSVRKKKPALKKIKSSTSVQSSATPPS
NTSSNPDIKCSNCTTSTTPLWRKDPKGLPLCNACGLFLKLHGVTRPLSLK
TDIIKKRQRSSTKINNNITPPPSSSLNPGAAGKKKNYTASVAASKRKNSL
NIVAPLKSQDIPIPKIASPSIPQYLRSNTRHHLSSSVPIEAETFSSFRPD
MNMTMNMNLHNASTSSFNNEAFWKPLDSAIDHHSGDTNPNSNMNTTPNGN
LSLDWLNLNLGPKKKRKVAPPTDVSLGDELHLDGEDVAMAHADALDDFDL
DMLGDGDSPGPGFTPHDSAPYGALDMADFEFEQMFTDALGIDEYGG
(Gat1 [1-510]; SV40 [512-518]; VP16 [519-596]).

In some embodiments, the synthetic TF comprises the indicated domains linked in the following order from N- to C-terminus: Gat1-TF1a-SV40-VP16. In one particular empbodiment, the synthetic TF comprises the following amino acid sequence:

(SEQ ID NO: 22)
MSNPDIKCSNCTTSTTPLWRKDPKGLPLCNACGLFLKLHGVTRPLSLKTD
IIKKRGPKKKRKVAPPTDVSLGDELHLDGEDVAMAHADALDDFDLDMLGD
GDSPGPGFTPHDSAPYGALDMADFEFEQMFTDALGIDEYGG
(TF1a [2-55], (304-357 of native seq);
SV40 [57-63]; VP16 [64-141]).

In some embodiments, the synthetic TF comprises the indicated domains linked in the following order from N- to C-terminus: Gat1-TF1b-SV40-VP16. In one particular empbodiment, the synthetic TF comprises the following amino acid sequence:

(SEQ ID NO: 23)
MPSNTSSNPDIKCSNCTTSTTPLWRKDPKGLPLCNACGLFLKLHGVTRPL
SLKTDIIKKRQRSSTKGPKKKRKVAPPTDVSLGDELHLDGEDVAMAHADA
LDDFDLDMLGDGDSPGPGFTPHDSAPYGALDMADFEFEQMFTDALGIDEY
GG
(TF1b [2-66], (299-363 of native seq);
SV40 [68-74]; VP16 [75-152]).

In some embodiments, the synthetic TF comprises the indicated domains linked in the following order from N- to C-terminus: Gat1-SV40-C1. In one particular empbodiment, the synthetic TF comprises the following amino acid sequence:

(SEQ ID NO: 24)
MHVFFPLLFRPSPVLFIACAYIYIDIYIHCTRCTVVNITMSTNRVPNLDP
DLNLNKEIWDLYSSAQKILPDSNRILNLSWRLHNRTSFHRINRIMQHSNS
IMDFSASPFASGVNAAGPGNNDLDDTDTDNQQFFLSDMNLNGSSVFENVF
DDDDDDDDVETHSIVHSDLLNDMDSASQRASHNASGFPNFLDTSCSSSFD
DHFIFTNNLPFLNNNSINNNHSHNSSHNNNSPSIANNTNANTNTNTSAST
NTNSPLLRRNPSPSIVKPGSRRNSSVRKKKPALKKIKSSTSVQSSATPPS
NTSSNPDIKCSNCTTSTTPLWRKDPKGLPLCNACGLFLKLHGVTRPLSLK
TDIIKKRQRSSTKINNNITPPPSSSLNPGAAGKKKNYTASVAASKRKNSL
NIVAPLKSQDIPIPKIASPSIPQYLRSNTRHHLSSSVPIEAETFSSFRPD
MNMTMNMNLHNASTSSFNNEAFWKPLDSAIDHHSGDTNPNSNMNTTPNGN
LSLDWLNLNLGPKKKRKVCETGQNSAAHRADPDSAGTTTTSAAAVWAPKA
VRCTGGLFFFHRDTTPAHAGETATPMAGGGGGGGGEAGSSDDCSSAASVS
LRVGSHDEPCFSGDGDGDWMDDVRALASFLESDEDWLRCQTA
(Gat1 [1-510]; SV40 [512-518]; C1 [519-642]).

In some embodiments, the synthetic TF comprises the indicated domains linked in the following order from N- to C-terminus: Gat1-TF1a-SV40-C1. In one particular empbodiment, the synthetic TF comprises the following amino acid sequence:

(SEQ ID NO: 25)
MSNPDIKCSNCTTSTTPLWRKDPKGLPLCNACGLFLKLHGVTRPLSLKTD
IIKKRGPKKKRKVCETGQNSAAHRADPDSAGTTTTSAAAVWAPKAVRCTG
GLFFFHRDTTPAHAGETATPMAGGGGGGGGEAGSSDDCSSAASVSLRVGS
HDEPCFSGDGDGDWMDDVRALASFLESDEDWLRCQTA
(TF1a [2-55], (304-357 of native seq);
SV40 [57-63]; C1 [64-187]).

In some embodiments, the synthetic TF comprises the indicated domains linked in the following order from N- to C-terminus: Gat1-TF1b-SV40-C1. In one particular empbodiment, the synthetic TF comprises the following amino acid sequence:

(SEQ ID NO: 26)
MPSNTSSNPDIKCSNCTTSTTPLWRKDPKGLPLCNACGLFLKLHGVTRPL
SLKTDIIKKRQRSSTKGPKKKRKVCETGQNSAAHRADPDSAGTTTTSAAA
VWAPKAVRCTGGLFFFHRDTTPAHAGETATPMAGGGGGGGGEAGSSDDCS
SAASVSLRVGSHDEPCFSGDGDGDWMDDVRALASFLESDEDWLRCQTA
(TF1b [2-66], (299-363 of native seq);
SV40 [68-74]; C1 [75-198]).

The amino acid sequence of MCM1 is as follows:

(SEQ ID NO: 27)
MSDIEEGTPTNNGQQKERRKIEIKFIENKTRRHVTFSKRKHGIMKKAFEL
SVLTGTQVLLLVVSETGLVYTFSTPKFEPIVTQQEGRNLIQACLNAPDDE
EEDEEEDGDDDDDDDDDGNDMQRQQPQQQQPQQQQQVLNAHANSLGHLNQ
DQVPAGALKQEVKSQLLGGANPNQNSMIQQQQHHTQNSQPQQQQQQQPQQ
QMSQQQMSQHPRPQQGIPHPQQSQPQQQQQQQQQLQQQQQQQQQQPLTGI
HQPHQQAFANAASPYLNAEQNAAYQQYFQEPQQGQY.

The amino acid sequence of MATAL1 is as follows:

(SEQ ID NO: 28)
MDDICSMAENINRTLFNILGTEIDEINLNTNNLYNFIMESNLTKVEQHTL
HKNISNNRLEIYHHIKKEKSPKGKSSISPQARAFLEQVFRRKQSLNSKEK
EEVAKKCGITPLQVRVWFINKRMRSK.

The amino acid sequence of MATAL2 is as follows:

(SEQ ID NO: 29)
MNK1PIKDLLNPQITDEFKSSILDINKKLFSICCNLPKLPESVTTEEEVE
LRDILGFLSRANKNRKISDEEKKLLQTTSQLTTTITVLLKEMRSIENDRS
NYQLTQKNKSADGLVFNVVTQDMINKSTKPYRGHRFTKENVRILESWFAK
NIENPYLDTKGLENLMKNTSLSRIQIKNWVSNRRRKEKTITIAPELADLL
SGEPLAKKKE.

The amino acid sequence of Yap1 is as follows:

(SEQ ID NO: 30)
MSVSTAKRSLDVVSPGSLAEFEGSKSRHDEIENEHRRTGTRDGEDSEQPK
KKGSKTSKKQDLDPETKQKRTAQNRAAQRAFRERKERKMKELEKKVQSLE
SIQQQNEVEATFLRDQLITLVNELKKYRPETRNDSKVLEYLARRDPNLHF
SKNNVNHSNSEPIDTPNDDIQENVKQKMNFTFQYPLDNDNDNDNSKNVGK
QLPSPNDPSHSAPMPINQTQKKLSDATDSSSATLDSLSNSNDVLNNTPNS
STSMDWLDNVIYTNRFVSGDDGSNSKTKNLDSNMFSNDFNFENQFDEQVS
EFCSKMNQVCGTRQCPIPKKPISALDKEVFASSSILSSNSPALTNTWESH
SNITDNTPANVIATDATKYENSFSGFGRLGFDMSANHYVVNDNSTGSTDS
TGSTGNKNKKNNNNSDDVLPFISESPFDMNQVTNFFSPGSTGIGNNAASN
TNPSLLQSSKEDIPFINANLAFPDDNSTNIQLQPFSESQSQNKFDYDMFF
RDSSKEGNNLFGEFLEDDDDDKKAANMSDDESSLIKNQLINEEPELPKQY
LQSVPGNESEISQKNGSSLQNADKINNGNDNDNDNDVVPSKEGSLLRCSE
IWDRITTHPKYSDIDVDGLCSELMAKAKCSERGVVINAEDVQLALNKHM
N.

The amino acid sequence of Gat1 is as follows:

(SEQ ID NO: 31)
MHVFFPLLFRPSPVLFIACAYIYIDIYIHCTRCTVVNITMSTNRVPNLDP
DLNLNKEIWDLYSSAQKILPDSNRILNLSWRLHNRTSFHRINRIMQHSNS
IMDFSASPFASGVNAAGPGNNDLDDTDTDNQQFFLSDMNLNGSSVFENVF
DDDDDDDDVETHSIVHSDLLNDMDSASQRASHNASGFPNFLDTSCSSSFD
DHFIFTNNLPFLNNNSINNNHSHNSSHNNNSPSIANNTNANTNTNTSAST
NTNSPLLRRNPSPSIVKPGSRRNSSVRKKKPALKKIKSSTSVQSSATPPS
NTSSNPDIKCSNCTTSTTPLWRKDPKGLPLCNACGLFLKLHGVTRPLSLK
TDIIKKRQRSSTKINNNITPPPSSSLNPGAAGKKKNYTASVAASKRKNSL
NIVAPLKSQDIPIPKIASPSIPQYLRSNTRHHLSSSVPIEAETFSSFRPD
MNMTMNMNLHNASTSSFNNEAFWKPLDSAIDHHSGDTNPNSNMNTTPNGN
LSLDWLNLNL.

The present invention also provides for a nucleic acid encoding any one of the synthetic TF of the present invention operatively linked to a promoter capable of expressing the synthetic TF in vitro or in vivo.

The present invention also provides for a vector comprising the nucleic acid of the present invention. In some embodiments, the vector is capable of stably integrating into a chromosome of a host cell or stably residing in a host cell. In some embodiments, the vector is an expression vector.

The present invention also provides for a host cell comprising the vector of the present invention, wherein the host cell is capable of expressing the synthetic TF.

The present invention also provides for a system comprising a nucleic acid of claim 55 and a second nucleic acid, or the nucleic acid, encodes a gene of interest (GOI) operatively linked to a promoter and one or more activator/repressor binding domains, or combination thereof, wherein the synthetic TF binds at least one of the one or more activator/repressor binding domain such that the synthetic TF modulates the expression of the GOI.

The present invention also provides for a genetically modified eukaryotic cell or organism, such as a plant cell or plant, comprising: (a) (i) one or more nucleic acids each encoding one or more transcription activators operatively linked to a first promoter, (ii) one or more nucleic acids each encoding one or more transcription repressors each operatively linked to a second promoter, or (iii) combinations thereof; and (b) one or more nucleic acids each encoding one or more independent genes of interest (GOI) each operatively linked to a promoter that is activated by the one or more transcription activators, repressed by the one or more transcription repressors, or a combination of both; wherein at least one transcription activator or transcription repressor is a synthetic transcription factor (TF) of claim 1.

In some embodiments, the first promoter, the second promoter, or both, is a tissue-specific or inducible promoter.

In some embodiments, the transcription activator is the synthetic TF. In some embodiments, the transcription repressor is the synthetic TF.

In some embodiments, any domain of the synthetic TF is heterologous to the plant cell or plant, one or more of the GOI, any other transcription activator or transcription repressor, and/or any of the promoters.

In some embodiments, the transcription activator is heterologous to the eukaryotic cell or organism, such as a plant cell or plant, one or more of the GOI, any other or transcription activator, transcription repressor, and/or any of the promoters. In some embodiments, the transcription repressor is heterologous to the eukaryotic cell or organism, such as a plant cell or plant, one or more of the GOI, any other transcription activator, and/or any of the promoters.

In some embodiments, the genetically modified eukaryotic cell or organism, such as a plant cell or plant comprises: (a) a first nucleic acid encoding a transcription activator operatively linked to a first tissue-specific or inducible promoter, (b) optionally a second nucleic acid encoding a transcription repressor operatively linked to a second tissue-specific or inducible promoter; and (c) one or more nucleic acids each encoding one or more independent genes of interest (GOI) each operatively linked to a promoter that is activated by the transcription activators, repressed by the transcription repressors, or a combination of both.

In some embodiments, the genetically modified eukaryotic cell or organism, such as a plant cell or plant comprises: (a) optionally a first nucleic acid encoding a transcription activator operatively linked to a first tissue-specific or inducible promoter, (b) a second nucleic acid encoding a transcription repressor operatively linked to a second tissue-specific or inducible promoter; and (c) one or more nucleic acids each encoding one or more independent genes of interest (GOI) each operatively linked to a promoter that is activated by the transcription activators, repressed by the transcription repressors, or a combination of both.

In some embodiments, the promoter is a tissue-specific promoter. Examples of tissue-specific promoters under developmental control include promoters that initiate transcription only (or primarily only) in certain tissues, such as vegetative tissues, cell walls, including e.g., roots or leaves. A variety of promoters specifically active in vegetative tissues, such as leaves, stems, roots and tubers are known. For example, promoters controlling patatin, the major storage protein of the potato tuber, can be used (see, e.g., Kim, Plant Mol. Biol. 26:603-615, 1994; Martin, Plant J. 11:53-62, 1997). The ORF13 promoter from Agrobacterium rhizogenes that exhibits high activity in roots can also be used (Hansen, Mol. Gen. Genet. 254:337-343, 1997). Other useful vegetative tissue-specific promoters include: the tarn promoter of the gene encoding a globulin from a major taro (Colocasia esculenta L. Schott) corm protein family, tarin (Bezerra, Plant Mol. Biol. 28:137-144, 1995); the curculin promoter active during taro corm development (de Castro, Plant Cell 4:1549-1559, 1992) and the promoter for the tobacco root-specific gene TobRB7, whose expression is localized to root meristem and immature central cylinder regions (Yamamoto, Plant Cell 3:371-382, 1991).

Leaf-specific promoters, such as the ribulose biphosphate carboxylase (RBCS) promoters can be used. For example, the tomato RBCS1, RBCS2 and RBCS3A genes are expressed in leaves and light-grown seedlings, only RBCS1 and RBCS2 are expressed in developing tomato fruits (Meier, FEBS Lett. 415:91-95, 1997). A ribulose bisphosphate carboxylase promoters expressed almost exclusively in mesophyll cells in leaf blades and leaf sheaths at high levels (e.g., Matsuoka, Plant J. 6:311-319, 1994), can be used. Another leaf-specific promoter is the light harvesting chlorophyll a/b binding protein gene promoter (see, e.g., Shiina, Plant Physiol. 115:477-483, 1997; Casal, Plant Physiol. 116:1533-1538, 1998). The Arabidopsis thaliana myb-related gene promoter (Atmyb5) (Li, et al., FEBS Lett. 379:117-121 1996), is leaf-specific. The Atmyb5 promoter is expressed in developing leaf trichomes, stipules, and epidermal cells on the margins of young rosette and cauline leaves, and in immature seeds. Atmyb5 mRNA appears between fertilization and the 16 cell stage of embryo development and persists beyond the heart stage. A leaf promoter identified in maize (e.g., Busk et al., Plant J. 11:1285-1295, 1997) can also be used.

Another class of useful vegetative tissue-specific promoters are meristematic (root tip and shoot apex) promoters. For example, the “SHOOTMERISTEMLESS” and “SCARECROW” promoters, which are active in the developing shoot or root apical meristems, (e.g., Di Laurenzio, et al., Cell 86:423-433, 1996; and, Long, et al., Nature 379:66-69, 1996); can be used. Another useful promoter is that which controls the expression of 3-hydroxy-3-methylglutaryl coenzyme A reductase HMG2 gene, whose expression is restricted to meristematic and floral (secretory zone of the stigma, mature pollen grains, gynoecium vascular tissue, and fertilized ovules) tissues (see, e.g., Enjuto, Plant Cell. 7:517-527, 1995). Also useful are knl-related genes from maize and other species which show meristem-specific expression, (see, e.g., Granger, Plant Mol. Biol. 31:373-378, 1996; Kerstetter, Plant Cell 6:1877-1887, 1994; Hake, Philos. Trans. R. Soc. Lond. B. Biol. Sci. 350:45-51, 1995). For example, the Arabidopsis thaliana KNAT1 promoter (see, e.g., Lincoln, Plant Cell 6:1859-1876, 1994) can be used.

In some embodiments, the promoter is substantially identical to the native promoter of a promoter that drives expression of a gene involved in secondary wall deposition. Examples of such promoters are promoters from IRX1, IRX3, IRX5, IRX8, IRX9, IRX14, IRX7, IRX10, GAUT13, or GAUT14 genes. Specific expression in fiber cells can be accomplished by using a promoter such as the NST1 promoter and specific expression in vessels can be accomplished by using a promoter such as VND6 or VND7. (See, e.g., PCT/US2012/023182 for illustrative promoter sequences). In some embodiments, the promoter is a secondary cell wall-specific promoter or a fiber cell-specific promoter. In some embodiments, the promoter is from a gene that is co-expressed in the lignin biosynthesis pathway (phenylpropanoid pathway). In some embodiments, the promoter is a C4H, C3H, HCT, CCR1, CAD4, CADS, FSH, PAL1, PAL2, 4CL1, or CCoAMT promoter. In some embodiments, the tissue-specific secondary wall promoter is an IRX1, IRX3, IRX5, IRX8, IRX9, IRX14, IRX7, IRX10, GAUT13, GAUT14, or CESA4 promoter. Suitable tissue-specific secondary wall promoters, and other transcription factors, promoters, regulatory systems, and the like, suitable for this present invention are taught in U.S. Patent Application Pub. Nos. 2014/0298539, 2015/0051376, and 2016/0017355.

One of skill will recognize that a tissue-specific promoter may drive expression of operably linked sequences in tissues other than the target tissue. Thus, as used herein a tissue-specific promoter is one that drives expression preferentially in the target tissue, but may also lead to some expression in other tissues as well.

In some embodiments, each GOI is operatively linked to a promoter that is activated by the transcription activator, repressed by the transcription repressors, or a combination of both.

In some embodiments, the promoter comprises one or more DNA-binding sites specific for the transcription activator, one or more DNA-binding sites specific for the transcription repressor, or a combination of both.

In some embodiments, the promoter comprises 1, 2, 3, 4, 5, 6, 7, 8, 9, or 10 DNA-binding sites specific for the transcription activator), 1, 2, 3, 4, 5, 6, 7, 8, 9, or 10 DNA-binding sites specific for the transcription repressor, or a combination of both.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1A: Design and characterization of a library of synthetic promoters. Brute force strategy to design and generate a library of promoters with varying expression strengths. Synthetic activators are generated by fusing a Gal4 DNA-binding domain to a nuclear localization sequence and VP16 activator domain. A library of cis-elements that bind Gal4 and vary in sequence were gathered from endogenous yeast promoters that fall within the Gal regulon. Various plant minimal promoters were also gathered. Five random cis-elements were concatenated in front of a minimal promoter in order to design and generate synthetic promoters.

FIG. 1B: Design and characterization of a library of synthetic promoters. Synthetic promoters were characterized by fusing them in front of a GFP. A constitutive MAS promoter was used to drive a DsRed in order to normalize between samples. Synthetic activators were driven by the constitutive Actin promoter, enabling the expression of the GFP. A control construct was generated, lacking the synthetic activator, allowing the measurement of basal expression of the synthetic promoters.

FIG. 1C: Design and characterization of a library of synthetic promoters. A range of expression strengths can be observed with the designed synthetic promoters. Constructs including the synthetic activator enable GFP expression (blue), while controls lacking synthetic activators provide basal expression levels of synthetic promoters (red). Constructs were transiently expressed in N. benthamiana leaves, and GFP fluorescence was normalized to constitutive expression of DsRed and reported in arbitrary units.

FIG. 2A. Utilizing synthetic promoters for the coordinated expression of multiple stacked transgenes. Schematic of how synthetic activator can be utilized to drive the concerted expression of multiple downstream genes of interest in a spatial or temporal specific manner. Cartoon demonstrates the basic design of constructs used to demonstrate how the expression of multiple transgenes (GFP, DsRed, and GUS) can be controlled by regulation of the synthetic activator.

FIG. 2B. Utilizing synthetic promoters for the coordinated expression of multiple stacked transgenes. Spatial regulation of multiple reporter genes under the control of the seed-specific expression of the synthetic activator driven by the At2S3 promoter. Seeds from transgenic plants show expression of all three reporter genes, whereas vegetative tissue taken from roots or whole seedling showed indication of reporter gene expression.

FIG. 2C. Utilizing synthetic promoters for the coordinated expression of multiple stacked transgenes. Temporal regulation of multiple reporter genes under the control of the synthetic activator which responds to environmental stimuli. The AtPht1.1 promoter is driving the synthetic activator, enabling the inducible expression of all three downstream reporter transgenes to be turned on in response to phosphate deprivation.

FIG. 3A. Teasing apart the additive effect of DNA elements reveals generalizable trends in promoter expression strength. Overall design of every combination of concatenated cis-elements and minimal promoters in order to test if individual parts additively contribute to expression strength in a predictable manner. Therefore, expected promoter strengths will increase with the addition of strong cis-elements and minimal promoters, whereas weak expression would be expected when using weak cis-elements and minimal promoters.

FIG. 3B. Teasing apart the additive effect of DNA elements reveals generalizable trends in promoter expression strength. Characterization of every combination of synthetic promoters generated from five cis-elements and five minimal promoters spanning expression strengths. Promoter strength is measured based on GFP fluorescence and normalized to constitutively expressed DsRed, in the same construct design described in FIGS. 1A to 1C. The general tendency of strong expression being correlated with the usage of DNA elements that promote expression is observed; however, some noise is still observed, which can be due to the context dependence of promoter elements.

FIG. 3C. Teasing apart the additive effect of DNA elements reveals generalizable trends in promoter expression strength. Qualitative characterization of five synthetic promoters demonstrating the trend of increasing promoter strength with usage of DNA elements with cis-elements and minimal promoters that promote higher expression. Five constructs (labeled and outlined in yellow) which span expression strengths varying expression strengths were infiltrated into a N. benthamiana leaf and imaged.

FIG. 4A. Implementation of cis and trans-element variations provides a wider range of expression strengths versus cis-elements alone. Combining randomly-concatenated cis-elements with various transcription factor designs to generate a larger and more diverse parts library of synthetic promoter/transcription factor pairs than with cis-elements alone.

FIG. 4B. Implementation of cis and trans-element variations provides a wider range of expression strengths versus cis-elements alone. Normalized distribution of expression strengths measured for both the cis-element library, and the cis and trans-element library. Bins were determined using the Freedman-Diaconis rule. A kernel density estimation for each parts library is displayed to highlight differences in the range of expression strengths of the two parts libraries.

FIG. 4C. Implementation of cis and trans-element variations provides a wider range of expression strengths versus cis-elements alone. A stacked barplot of the unique synthetic promoter/transcription factor pairs of the two libraries and their measured expression strengths. Bars are stacked such that each bin displays the contribution of a given library to the overall bin count.

FIG. 5A. Utilizing synthetic repressors enable synthetic promoter compatibility with repressor logic. Schematic of constructs used to demonstrate how the synthetic repressor inhibits and competes with the synthetic activator to repress expression of a given transgene. Samples above the grey dashed line are only expressing the synthetic activator, whereas samples below the dashed line have included expression of the synthetic repressor.

FIG. 5B. Utilizing synthetic repressors enable synthetic promoter compatibility with repressor logic. Labeling the various constructs infiltrated into a leaf with and without the synthetic repressor. Two different synthetic promoters (one high and one medium expression strength) were used to demonstrate the effects of the synthetic repressor. DsRed expression is constitutively driven by the nos promoter to enable normalization. Infiltration of a construct only expressing the synthetic repressor was used as a negative control.

FIG. 5C. Utilizing synthetic repressors enable synthetic promoter compatibility with repressor logic. GFP expression controlled by two different synthetic promoters. Spots infiltrated without the repressor are above the grey dashed line, whereas the synthetic repressor was also co-infiltrated in samples below the dashed line. Samples within dashed yellow lines correspond to each other as with and without the synthetic repressor.

FIG. 5D. Utilizing synthetic repressors enable synthetic promoter compatibility with repressor logic. Constitutive expression of DsRed allows for an internal control and normalization of GFP expression, as it is generally expressed at the same level in all samples.

FIG. 5E. Utilizing synthetic repressors enable synthetic promoter compatibility with repressor logic. Quantification of the amount of repression observed with the introduction of the synthetic repressor in conjunction with constructs already expressing a synthetic activator driving GFP expression. Samples are normalized to DsRed expression.

FIG. 5F. Utilizing synthetic repressors enable synthetic promoter compatibility with repressor logic. Repression of leaky MCM1 promoters with addition of a synthetic repressor to MCM1. Samples are normalized to mCherry expression.

FIG. 6A. Building a heterologous synthetic activator-promoter system using the GAL4 network for plant expression. Rationale of utilizing endogenous cis-elements from Gal promoters in the Saccharomyces Gal regulon. Various UAS/Gal cis-elements identified from varying Saccharomyces Gal promoter regions.

FIG. 6B. Building a heterologous synthetic activator-promoter system using the GAL4 network for plant expression. Various cis-elements (UAS) are used to build a randomized cis-element library based on concatenation of five variants. These are fused to a library of plant minimal promoters, building a library of synthetic promoters that replicate the endogenous yeast Gal regulon. These promoters are then characterized and tested in plants as an orthogonal synthetic transcription system for controlling gene expression strength and tissue-specificity of stacked transgenes.

FIG. 7A. Expression strengths of synthetic promoters designed to investigate the rational design and contributory effects of combinations of cis-elements. Name of all constructs tested corresponding to the UAS and minimal promoters fused together to build each synthetic promoter. Heatmap corresponds to FIG. 3B.

FIG. 7B. Expression strengths of synthetic promoters designed to investigate the rational design and contributory effects of combinations of cis-elements. Corresponding expression strength of all characterized promoters. Values correspond to the expression strength.

FIG. 8A. Design and characterization of different repressor constructs. Various synthetic repressor fusion constructs were tested to optimize transcriptional repression. SRDX repression domain was fused to both C- and N-termini, and with and without the VP16 domain.

FIG. 8B. Design and characterization of different repressor constructs. One synthetic promoter was expressed with the Gal4 synthetic activator, then co-infiltrated with the three different repressor constructs, which yielded a decrease in GFP expression, normalized to DsRed. Construct pms6384 repressed gene expression the best for both synthetic promoters and was characterized as the most efficient synthetic repressor tested.

FIG. 8C. Design and characterization of different repressor constructs. Another different synthetic promoter was expressed with the Gal4 synthetic activator, then co-infiltrated with the three different repressor constructs, which yielded a decrease in GFP expression, normalized to DsRed. Construct pms6384 repressed gene expression the best for both synthetic promoters and was characterized as the most efficient synthetic repressor tested.

FIG. 9. Design and nomenclature of synthetic transcription factors for the cis+trans-element library. Synthetic trans-element design and nomenclature follow the depicted pipeline. Transcription factors are initially selected as either full-length WT or only DNA-binding domain and subsequently fused with a selection of NLS, TAD, or repressor domains.

FIG. 10. Expression strengths of synthetic promoter and transcription factor pairs with GatI transcription factor system. Combinations of Gatl TF/promoter corresponds to a subset of pairs shown in FIGS. 4A to 4C. Expression strength is calculated from dividing GFP fluorescence from a control mCherry fluorescence.

FIG. 11. Expression strengths of synthetic promoter and transcription factor pairs with MATAL1 transcription factor system. Combinations of MATAL1 TF/promoter corresponds to a subset of pairs shown in FIGS. 4A to 4C. Expression strength is calculated from dividing GFP fluorescence from a control mCherry fluorescence.

FIG. 12. Expression strengths of synthetic promoter and transcription factor pairs with MATAL2 transcription factor system. Combinations of MATAL2 TF/promoter corresponds to a subset of pairs shown in FIGS. 4A to 4C. Expression strength is calculated from dividing GFP fluorescence from a control mCherry fluorescence.

FIG. 13. Expression strengths of synthetic promoter and transcription factor pairs with MCM1 transcription factor system. Combinations of MCM1 TF/promoter corresponds to a subset of pairs shown in FIGS. 4A to 4C. Expression strength is calculated from dividing GFP fluorescence from a control mCherry fluorescence.

FIG. 14. Expression strengths of synthetic promoter and transcription factor pairs with Yap1 transcription factor system. Combinations of Yap1 TF/promoter corresponds to a subset of pairs shown in FIGS. 4A to 4C. Expression strength is calculated from dividing GFP fluorescence from a control mCherry fluorescence.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The term “about” refers to a value including 10% more than the stated value and 10% less than the stated value.

As used herein, the term “promoter” refers to a polynucleotide sequence capable of driving transcription of a DNA sequence in a cell. Thus, promoters used in the polynucleotide constructs of the invention include cis- and trans-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. Promoters are located 5′ to the transcribed gene, and as used herein, include the sequence 5′ from the translation start codon.

A “constitutive promoter” is one that is capable of initiating transcription in nearly all cell types, whereas a “cell type-specific promoter” initiates transcription only in one or a few particular cell types or groups of cells forming a tissue. In some embodiments, the promoter is secondary cell wall-specific and/or fiber cell-specific. A “fiber cell-specific promoter” refers to a promoter that initiates substantially higher levels of transcription in fiber cells as compared to other non-fiber cells of the plant. A “secondary cell wall-specific promoter” refers to a promoter that initiates substantially higher levels of transcription in cell types that have secondary cell walls, e.g., lignified tissues such as vessels and fibers, which may be found in wood and bark cells of a tree, as well as other parts of plants such as the leaf stalk. In some embodiments, a promoter is fiber cell-specific or secondary cell wall-specific if the transcription levels initiated by the promoter in fiber cells or secondary cell walls, respectively, are at least 3-fold, 4-fold, 5-fold, 6-fold, 7-fold, 8-fold, 9-fold, 10-fold, 50-fold, 100-fold, 500-fold, 000-fold higher or more as compared to the transcription levels initiated by the promoter in other tissues, resulting in the encoded protein substantially localized in plant cells that possess fiber cells or secondary cell wall, e.g., the stem of a plant. Non-limiting examples of fiber cell and/or secondary cell wall specific promoters include the promoters directing expression of the genes IRX1, IRX3, IRX5, IRX7, IRX8, IRX9, IRX10, IRX14, NST1, NST2, NST3, MYB46, MYB58, MYB63, MYB83, MYB85, MYB103, PAL1, PAL2, C3H, CcOAMT, CCR1, FSH, LAC4, LAC17, CADc, and CADd. See, e.g., Turner et al 1997; Meyer et al 1998; Jones et al 2001; Franke et al 2002; Ha et al 2002;Rohde et al 2004; Chen et al 2005; Stobout et al 2005; Brown et al 2005; Mitsuda et al 2005; Zhong et al 2006; Mitsuda et al 2007; Zhong et al 2007a, 2007b; Zhou et al 2009; Brown et al 2009; McCarthy et al 2009; Ko et al 2009; Wu et al 2010; Berthet et al 2011. In some embodiments, a promoter is substantially identical to a promoter from the lignin biosynthesis pathway. A promoter originated from one plant species may be used to direct gene expression in another plant species.

A polynucleotide or amino acid sequence is “heterologous” to an organism or a second polynucleotide or amino acid sequence if it originates from a foreign species, or, if from the same species, is modified from its original form. For example, when a polynucleotide encoding a polypeptide sequence is said to be operably linked to a heterologous promoter, it means that the polynucleotide coding sequence encoding the polypeptide is derived from one species whereas the promoter sequence is derived from another, different species; or, if both are derived from the same species, the coding sequence is not naturally associated with the promoter (e.g., is a genetically engineered coding sequence, e.g., from a different gene in the same species, or an allele from a different ecotype or variety, or a gene that is not naturally expressed in the target tissue).

The term “operably linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a DNA or RNA sequence if it stimulates or modulates the transcription of the DNA or RNA sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The terms “host cell” of “host organism” is used herein to refer to a living biological cell that can be transformed via insertion of an expression vector.

The terms “expression vector” or “vector” refer to a compound and/or composition that transduces, transforms, or infects a host cell, thereby causing the cell to express nucleic acids and/or proteins other than those native to the cell, or in a manner not native to the cell. An “expression vector” contains a sequence of nucleic acids (ordinarily RNA or DNA) to be expressed by the host cell. Optionally, the expression vector also comprises materials to aid in achieving entry of the nucleic acid into the host cell, such as a virus, liposome, protein coating, or the like. The expression vectors contemplated for use in the present invention include those into which a nucleic acid sequence can be inserted, along with any preferred or required operational elements. Further, the expression vector must be one that can be transferred into a host cell and replicated therein. Particular expression vectors are plasmids, particularly those with restriction sites that have been well documented and that contain the operational elements preferred or required for transcription of the nucleic acid sequence. Such plasmids, as well as other expression vectors, are well known to those of ordinary skill in the art.

The terms “polynucleotide” and “nucleic acid” are used interchangeably and refer to a single or double-stranded polymer of deoxyribonucleotide or ribonucleotide bases read from the 5′ to the 3′ end. A nucleic acid of the present invention will generally contain phosphodiester bonds, although in some cases, nucleic acid analogs may be used that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); positive backbones; non-ionic backbones, and non-ribose backbones. Thus, nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

The synthetic TF of the present invention can be used in the invention taught in PCT International Patent Application No. PCT/US2018/050514 (Publication No. WO 2019/051503 A2), which is hereby incorporated by reference.

The present invention can be used in new or non-model organisms for the controlled expression of multiple genes in a certain manner, including expressing multiple genes simultaneously. The expression of these genes can be regulated in a temporal and/or spatial manner.

The present invention can be used in a strategy to design system utilizing synthetic promoters for the ultimate purpose of controlling expression strength, tissue-specificity, and environmentally-responsive promoters and associated downstream products (e.g. RNA, protein). This method utilizes the synthetic TF of the present invention with its corresponding DNA binding sequence (cis-element), where multiple slightly varying nucleotide sequences of cis-elements are concatenated to provide variability in the binding strength of the transcriptional regulator. The cis-elements are fused to varying minimal promoter sequences (minimal promoter or minimal promoter+UTR upstream sequence of ATG) of the eukaryote host organism of interest to enable the synthetic TF the ability to control expression of the target downstream gene. This invention provides a strategy for engineering an entirely orthogonal transcriptional network into any eukaryotic host for controlling expression strengths of multiple genes through the heterologous expression of the synthetic TF.

The present invention enables one skilled in the art to control the expression of a single or multiple genes simultaneously in any eukaryote organism with only one endogenous promoter using the synthetic TF. Many times, such as in plants, reuse of the same promoter to drive heterologous expression of multiple genes may increase the likelihood of gene silencing and even creates genome instability. Moreover, use of one endogenous promoter may offer the desired expression level required to express a gene of interest. The present invention offers the capacity of retaining expression specificity while offering a dynamic range of expression of the transgene using the synthetic TF. For example, there are many promoters that display tissue-specific expression in one specific tissue (e.g., plant roots, seeds, leaves, or the like). By utilizing a promoter of interest to drive expression of the synthetic TF, one can generate a library of synthetic promoters that are turned on by the synthetic TF at varying expression strengths. This is an efficient and productive way in controlling the exact expression strength of a single or multiple genes in a tissue-specific or environmentally-responsive manner.

The present invention can be applied to any host eukaryotic organism of interest, such as fungi, plant, and animal cells., using the synthetic TF. This invention offers the ability to perform various permutations and test multiple expression profiles. For example, one set of plants could be generated with different promoters driving the synthetic TF (set A) and another set of plants would be transformed with different combination of synthetic promoters driving one or a multiple transgene of interests (set B). Plants from set A could be crossed with those of set B, this would great a 2D matrix of new plants expressing transgene of interests in different tissues and at different strength. This approach has the capacity to reduce number of transformations. For example, generation of 50 plants for each set (A and B) will require 100 transformations and will be used to generate 2500 combinations that would normally require 2500 independent transformations without the use of matrix as presented above. Such matrix approach is applicable to any eukaryotic host that can be crossed such as crops and yeast.

The present invention provides for a strategy to repress genes of interest using the synthetic TF. The invention described here provides an additional layer of control and regulation by utilizing synthetic TF to repress expression of genes. The synthetic TF would comprise a DNA-binding domain which binds the synthetic promoter cis elements and a repressor domain. There are varying strategies to control the level of repression. Various derivatives of the synthetic TF (N- or C-terminus) can result in varying levels of repression. Furthermore, repressors could also either be degrade, sequestered, or change in protein conformation to control spatial and temporal changes in repression of genes of interest.

With the synthetic TF of this present invention, one skilled in the art is able to subtract out certain tissues for where one or more genes of interest (GOI) are expressed. For example, one can use a constitutive promoter to activate expression of GOls in all tissue and express a repressor specifically in the roots; thus, only expression will be found in the shoots. This is useful for those who may want to avoid the length and laborious process of discovering, characterizing, and validating promoters that have properties they want. Furthermore, within the context of the synthetic promoters system, this provides an additional level of regulation which other strategies and technologies do not have. A further application of this invention is in the context of an environmental response. For example, if one desires a GOl to be repressed in response to an abiotic or biotic stress for optimal growth, the present invention can provide for a repression system to effect a gradual decrease in expression of the GOls.

This invention can be used by nearly any biotechnology industry. This invention can easily be utilized for any eukaryotic host, such as plant, yeast or animal hosts.

The present invention provides for the following embodiments of the invention:

A synthetic transcription factor (TF) comprising (a) a DNA-binding domain of a transcription factor linked to (b) an activator domain or repressor domain, and (c) a nuclear localization sequence (NLS).

In some embodiments, the DNA-binding domain is a DNA-binding domain of a eukaryotic TF or a prokaryotic TF.

In some embodiments, the DNA-binding domain is a DNA-binding domain of a eukaryotic TF.

In some embodiments, the eukaryotic TF is a yeast TF.

In some embodiments, the yeast TF is a Saccharomyces TF.

In some embodiments, the Saccharomyces TF is a Saccharomyces cerevisiae TF.

In some embodiments, the S. cerevisiae TF is Gal4, YAP1, GAT1, MATAL1, MATAL2, MCM1, Abf1, Adr1, Ash1, Gcn4, Gcr1, Hap4, Hsf1, Ime1, Ino2/Ino4, Leu3, Lys14, Mata2, Mga2, Met4, Mig1, Rap1, Rgt1, Rlm1, Smp1, Rme1, Rox1, Rtg3, Spt23, Tea1, Ume6, or Zap1.

In some embodiments, the S. cerevisiae TF is Gal4, YAP1, GAT1, MATAL1, MATAL2, or MCM1.

In some embodiments, the S. cerevisiae TF is Gal4.

In some embodiments, the S. cerevisiae TF is YAP1.

In some embodiments, the S. cerevisiae TF is GAT1.

In some embodiments, the S. cerevisiae TF is MATAL1.

In some embodiments, the S. cerevisiae TF is MATAL2.

In some embodiments, the S. cerevisiae TF is MCM1.

In some embodiments, the S. cerevisiae TF is Rap1.

In some embodiments, the DNA-binding domain comprises the amino acid sequence of Rap1, or GXXIRXRF (wherein X is any amino acid) (SEQ ID NO:41), G(G, P, A or R)(S or A) IRXRF (wherein X is any amino acid) (SEQ ID NO:42), or GNSIRHRFRV (SEQ ID NO:43).

In some embodiments, the synthetic TF comprises the activator domain which is a herpes simplex virus VP16, maize C1, or a yeast activator domain.

In some embodiments, the activator domain is the yeast activator domain.

In some embodiments, the yeast activator domain is a Saccharomyces activator domain.

In some embodiments, the Saccharomyces activator domain is a Saccharomyces cerevisiae activator domain.

In some embodiments, the S. cerevisiae activator domain is a Gal4, YAP1, GAT1, MATAL1, MATAL2, MCM1, Abf1, Adr1, Ash1, Gcn4, Gcr1, Hap4, Hsf1, Ime1, Ino2/Ino4, Leu3, Lys14, Mga2, Met4, Rap1, Rlm1, Smp1, Rtg3, Spt23, Tea1, Ume6, or Zap1 activator domain.

In some embodiments, the S. cerevisiae activator domain is a Rap1 activator domain.

In some embodiments, the synthetic TF comprises the repressor domain.

In some embodiments, the repressor domain comprises an EAR motif, TLLLFR motif, R/KLFGV motif, LxLxPP motif, or a yeast repressor domain.

In some embodiments, the repressor domain comprises the EAR motif.

In some embodiments, the repressor domain comprises the TLLLFR motif.

In some embodiments, the repressor domain comprises the R/KLFGV motif.

In some embodiments, the repressor domain comprises the LxLxPP motif.

In some embodiments, the repressor domain is the yeast repressor domain.

In some embodiments, the yeast repressor domain is a Saccharomyces repressor domain.

In some embodiments, the Saccharomyces repressor domain is a Saccharomyces cerevisiae repressor domain.

In some embodiments, the S. cerevisiae repressor domain is an Ash1, Matα2, Mig1, Rap1, Rgt1, Rme1, Rox1, or Ume6 repressor domain.

In some embodiments, the NLS is monopartite.

In some embodiments, the NLS is bipartite.

In some embodiments, the NLS comprises a M9 domain or PY-NLS motif.

In some embodiments, the NLS comprises the amino acid sequence KIPIK (yeast Matα2).

In some embodiments, any two, or all, of the DNA-binding domain, the activator domain, the repressor domain, and the NLS are heterologous to each other.

In some embodiments, one or more, or all, of the DNA-binding domain, the activator domain, the repressor domain, and the NLS are obtained or derived from a non-viral organism.

In some embodiments, the DNA-binding domain, the NLS, and the activator domain or repressor domain are linked in this order from N- to C-terminus.

A nucleic acid encoding the synthetic TF of any one of claims 1-54 operatively linked to a promoter capable of expressing the synthetic TF in vitro or in vivo.

A vector comprising the nucleic acid of the present invention.

In some embodiments, the vector is capable of stably integrating into a chromosome of a host cell or stably residing in a host cell.

In some embodiments, the vector is an expression vector.

A host cell comprising the vector of the present invention, wherein the host cell is capable of expressing the synthetic TF.

A system comprising a nucleic acid of the present invention and a second nucleic acid, or the nucleic acid, encodes a gene of interest (GOI) operatively linked to a promoter and one or more activator/repressor binding domains, or combination thereof, wherein the synthetic TF binds at least one of the one or more activator/repressor binding domain such that the synthetic TF modulates the expression of the GOI.

A genetically modified eukaryotic cell or organism, such as a plant cell or plant, comprising: (a) (i) one or more nucleic acids each encoding one or more transcription activators operatively linked to a first promoter, (ii) one or more nucleic acids each encoding one or more transcription repressors each operatively linked to a second promoter, or (iii) combinations thereof; and (b) one or more nucleic acids each encoding one or more independent genes of interest (GOI) each operatively linked to a promoter that is activated by the one or more transcription activators, repressed by the one or more transcription repressors, or a combination of both; wherein at least one transcription activator or transcription repressor is a synthetic transcription factor (TF) of the present invention.

In some embodiments, the first promoter, the second promoter, or both, is a tissue-specific or inducible promoter.

In some embodiments, the transcription activator is the synthetic TF.

In some embodiments, the transcription repressor is the synthetic TF.

In some embodiments, any domain of the synthetic TF is heterologous to the eukaryotic cell or organism, such as a plant cell or plant, one or more of the GOI, any other transcription activator or transcription repressor, and/or any of the promoters.

In some embodiments, the transcription activator is heterologous to the eukaryotic cell or organism, such as a plant cell or plant, one or more of the GOI, any other or transcription activator, transcription repressor, and/or any of the promoters.

In some embodiments, the transcription repressor is heterologous to the eukaryotic cell or organism, such as a plant cell or plant, one or more of the GOI, any other transcription activator, and/or any of the promoters.

In some embodiments, the genetically modified plant cell or plant comprises: (a) a first nucleic acid encoding a transcription activator operatively linked to a first tissue-specific or inducible promoter, (b) optionally a second nucleic acid encoding a transcription repressor operatively linked to a second tissue-specific or inducible promoter; and (c) one or more nucleic acids each encoding one or more independent genes of interest (GOI) each operatively linked to a promoter that is activated by the transcription activators, repressed by the transcription repressors, or a combination of both.

In some embodiments, the genetically modified plant cell or plant comprises: (a) optionally a first nucleic acid encoding a transcription activator operatively linked to a first tissue-specific or inducible promoter, (b) a second nucleic acid encoding a transcription repressor operatively linked to a second tissue-specific or inducible promoter; and (c) one or more nucleic acids each encoding one or more independent genes of interest (GOI) each operatively linked to a promoter that is activated by the transcription activators, repressed by the transcription repressors, or a combination of both.

In some embodiments, each GOI is operatively linked to a promoter that is activated by the transcription activator, repressed by the transcription repressors, or a combination of both.

In some embodiments, the promoter comprises one or more DNA-binding sites specific for the transcription activator, one or more DNA-binding sites specific for the transcription repressor, or a combination of both.

In some embodiments, the promoter comprises 1, 2, 3, 4, 5, 6, 7, 8, 9, or 10 DNA-binding sites specific for the transcription activator), 1, 2, 3, 4, 5, 6, 7, 8, 9, or 10 DNA-binding sites specific for the transcription repressor, or a combination of both.

In some embodiments, the eukaryotic cell or organism is a plant cell or plant.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### EXAMPLE 1

### Engineering and Strategic Design of an Orthogonal Transcription Regulation System to Modulate Gene Expression in Planta

Many strategies in agricultural biotechnology require the precise and coordinated control of multiple genes to effectively modify complex plant traits. Thus, engineering efforts have been hindered by the lack of characterized tools that allow for reliable and targeted expression of transgenes. Here we have designed and characterized a library of synthetic promoters to facilitate multi-gene engineering efforts that enable the control of expression level and tissue-specificity in planta. By leveraging an orthogonal transcriptional system, we have developed a strategy that utilizes both synthetic activators and synthetic repressors to coordinate the simultaneous expression of several genes, introducing logic principles for genetic circuits and multiple dimensions of transcriptional control. Moreover, our characterization of the contributory genetic elements that dictate gene expression provides the foundation for future efforts in the rational design of more refined synthetic promoter and synthetic activation/repressor elements for coordinated expression. Our findings demonstrate how these elements can enable the concerted expression of multiple genes simultaneously in a tissue-specific and environmental-responsive manner, providing the basis for more elegant and sophisticated plant engineering endeavors.

Our cis-elements are based on orthogonal transcription factors from Saccharomyces cerevisiae with which we generated synthetic DNA-binding cis-element sites through random concatenation of known transcription factor binding motifs, resulting in unique upstream activating sequences (UAS). We introduce these synthetic UAS constructs upstream of varying plant minimal promoters to generate synthetic promoters. These components generated a diversity of expression strengths up to 10-fold over background expression when coupled with a single synthetic trans-activator. Furthermore, we expanded the expression strengths of our library up to 50-fold by introducing trans-element diversity using modularly designed transcription factors containing activation, repression, and DNA-binding domains. We demonstrate this strategy in six orthogonal transcription factor systems, generating ˜300 promoter/transcription factor pairs of varying expression strengths. Additionally, we were able to confirm that our repressor constructs had the capacity to terminate the basal expression of numerous promoter elements. Furthermore, we demonstrate our synthetic promoter system is inducible and tissue specific in stable Arabidopsis lines.

A major obstacle that has often thwarted plant engineering efforts is the natural phenomenon of epigenetic silencing. Plants have evolved robust defense mechanisms that may perceive multiple transgenes driven by the same promoter as a threat, resulting in gene silencing at the transcriptional (gene inactivation via DNA methylation) and post-transcriptional (RNA degradation) leve18. Thus, although many engineering efforts require the coordinated expression of multiple genes, it has long been observed that stacking the same promoter multiple times may also dramatically increase the chance of gene silencing^{,}. Inherent to our library design of synthetic promoters is the avoidance of identical sequences, as addressed by the randomization of varying UAS combinations and minimal promoter sequences to avoid the use of homologous sequences. This strategy permits the stacking of multiple genes each with distinct promoters with the goal of circumventing potential gene silencing. Additionally, it is important to note that many of the classically used promoters have been incorrectly labeled as ‘constitutive,’ as they are not expressed in all tissue⁶, nor do they express at similar expression levels across various tissue types. Thus, this broad stroked approach and traditional reliance on ‘constitutive’ promoters represents a major barrier that separates the level of engineering complexity that can be deployed in model microbial versus plant systems.

Another key distinction between plants and unicellular microbes is the drastically different levels of organismal and developmental complexity, best demonstrated by the multitude of tissue types that make up plants. Additionally, many of these different cell types are highly dynamic and respond to their external environment. Thus, a key hurdle has been how to artificially design promoter elements that replicate elements of spatial and temporal control over gene expression^{,}. In comparison to microbial efforts, these challenges are unique and specific to plants and have not yet been thoroughly addressed. The majority of previous studies that have required tissue-specific expression of transgenes in plants have utilized endogenous promoters; however, these promoters cannot modulate expression strength, thus demonstrating the one-dimensional limitation of being restricted to these endogenous sequences. The use of synthetic trans-activators would allow for varying levels of transcription when expressed under an endogenous promoter and coupled with our synthetic promoters. The distinct output of each promoter will provide the capacity to express numerous trans-genes at varying strengths with a single synthetic trans-activator, providing an additional dimension of control over synthetic genetic circuits.

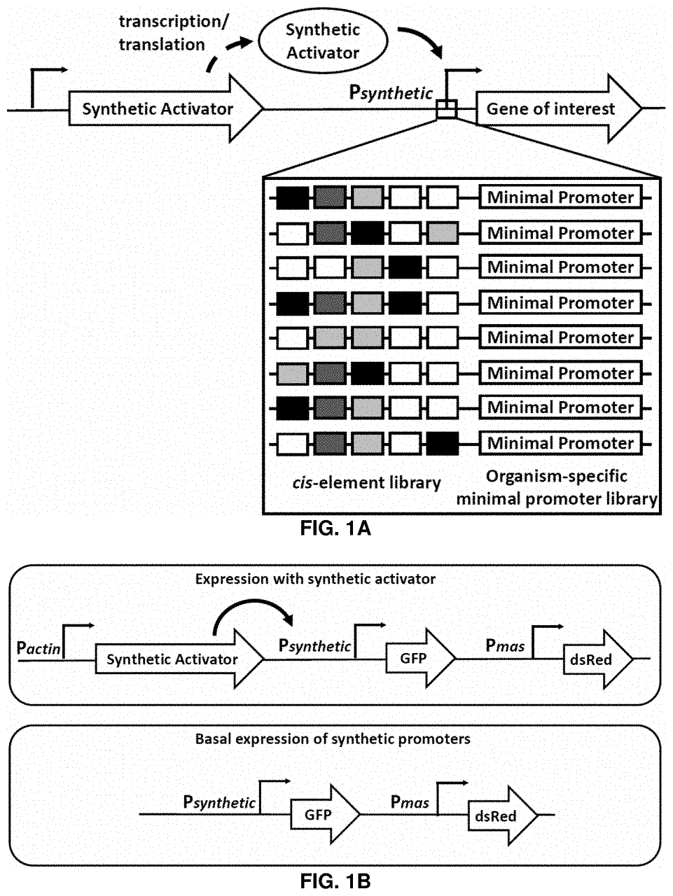

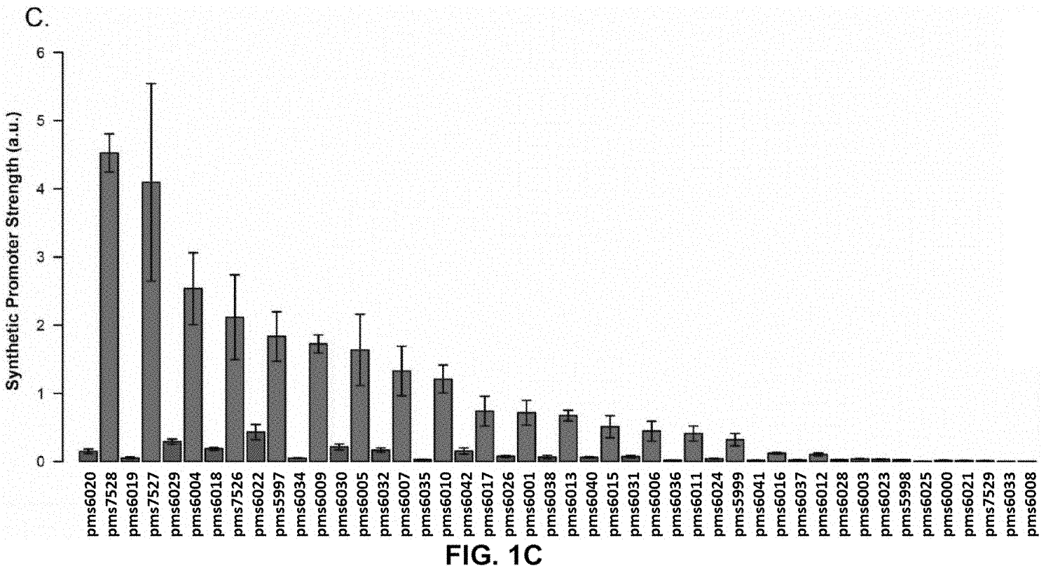

To develop a library of synthetic promoters that cover a wide range of expression strengths, we designed a system that would be turned on by an orthogonal transcription factor, hereon referred to as a synthetic trans-activator. We utilized the well-characterized transcription factor Gal4, from S. cerevisiae, fused to a VP16 activator domain as a synthetic trans-activator¹⁰. The heterologous nature of Ga14, originating from a distantly related organism, provides a means to leverage a purely orthogonal system in plants, separating the transcriptional regulation of introduced transgenes from endogenous genes. A diversity of known Gal4 cis-elements—also known as upstream activating sequences (UAS)—were taken from endogenous promoters in the yeast Gal regulon, displaying distinct nucleotide sequences and assumed to exhibit a diversity of dissociation constants with Gal4. Moreover, it has become increasingly recognized that the minimal promoter—i.e., the region in which RNA polymerase II is recruited via the TATA element to initiate transcription—also plays a key role in determining the expression strength of a given gene¹³. We generated a library of synthetic promoters in which each promoter was composed of five randomly concatenated Gal4 UASs upstream of a previously characterized plant minimal promoter^{11, 12} (FIGS. 1A to 1C).

Harnessing the diversity of our randomly-concatenated cis-element library coupled with a single trans-activator, a range of expression strengths were observed using Green Fluorescent Protein (GFP) fluorescence as a proxy for transcriptional output. A constitutive plant promoter, pMAS, was used to drive expression of dsRED and served as normalization agent, with the ratio of GFP over dsRED providing our “normal” value for the output of each synthetic trans-activator/promoter combination. The inherent variability in expression from leaf to leaf requires this normalization when comparing values from different biological replicates. As expected, we observed a wide distribution of expression strengths, with the resulting pool of promoters spanning a ten-fold range of normalized expression while avoiding the usage of identical sequences. (FIGS. 1A to 1C).

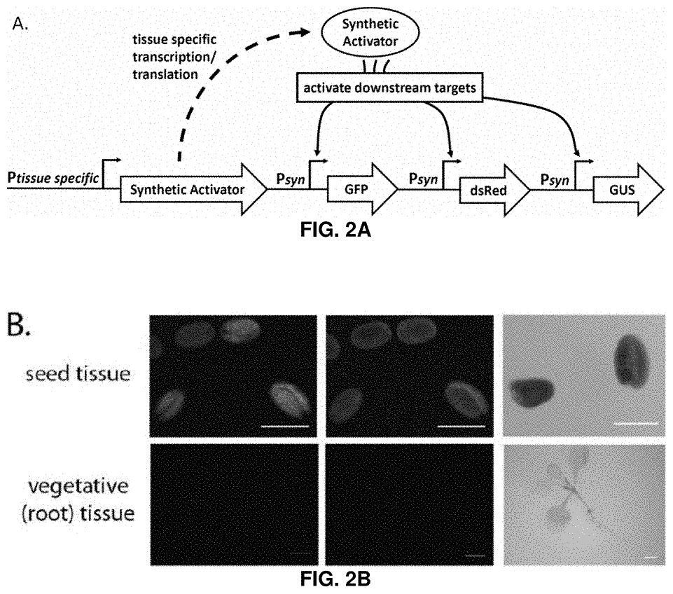

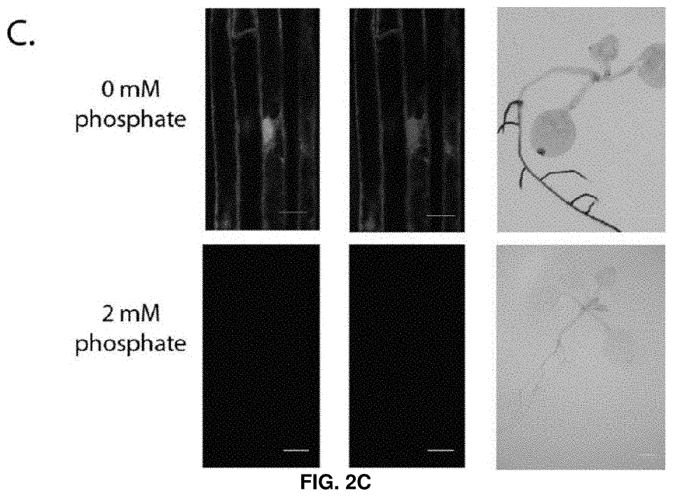

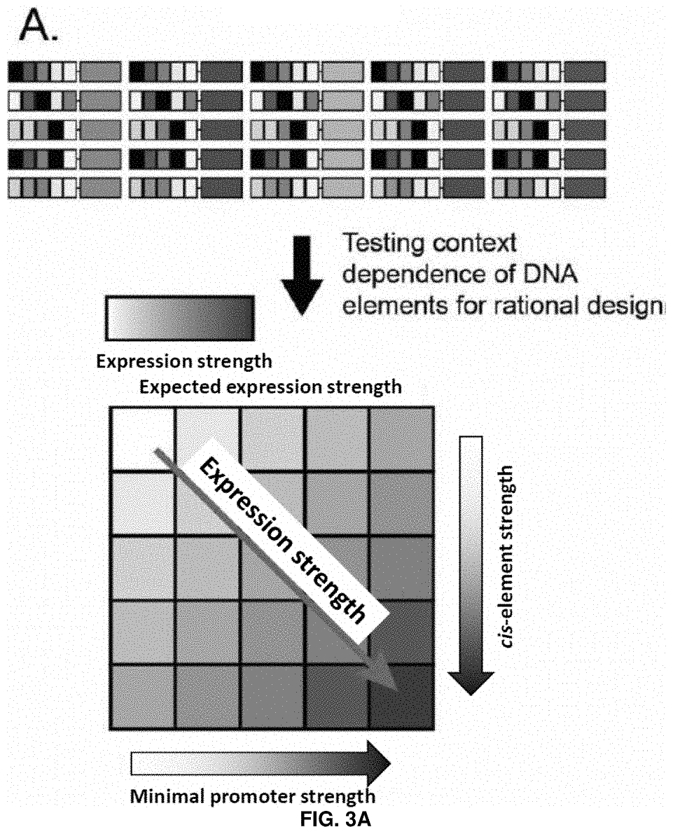

In order to demonstrate that these genes can coordinate the expression of multiple genes in a tissue-specific manner, we expressed the synthetic activator under a seed-specific promoter, stacked with three reporter genes (GFP, DsRed, and beta-glucuronidase (GUS)) driven by three different synthetic promoters. As the reporters can only be turned on by the synthetic activator, we observed the expected expression of all three reporters specifically in seed tissue (FIGS. 2A to 2C). Our findings demonstrate the use of synthetic promoters in the spatial regulation of multiple stacked genes simultaneously.

In many cases, it is not necessary to express transgenes constantly, and thus temporal control over gene expression provides an additional dimension of control over engineering endeavors. For example, the constant overexpression of a given protein in an organism may act as a sink on cellular resources resulting in overall detrimental effects¹⁵. Similarly, various agricultural traits (e.g., disease resistance) can many times result in fitness costs, and thus targeted expression of these genes may curtail unintended consequences^{16, 17}. Thus, coordinating the expression of multiple genes in an inducible or environmentally-responsive manner may provide a solution to many plant engineering efforts. In order to highlight how our synthetic promoters may address some of these issues, we designed a synthetic activator driven by a phosphate responsive promoter, which is induced under low external phosphate concentrations, again stacked with three reporter genes driven by synthetic promoters. Stable Arabidopsis transformants display the expression of all reporter genes in response to the absence of phosphate in media (FIGS. 2A to 2C), and as expected, reporter genes were repressed in the presence of phosphate. These results display how our synthetic promoters may be leveraged to enable fine-tuned control over stacked genes in applications that require complex engineering systems that can respond to environmental cues (i.e., abiotic and biotic stresses). This strategy can be further expanded by selecting a specific synthetic promoter for each trans-gene, allowing for a single environmental cue to initiate a series of transcriptional events of varying strength.

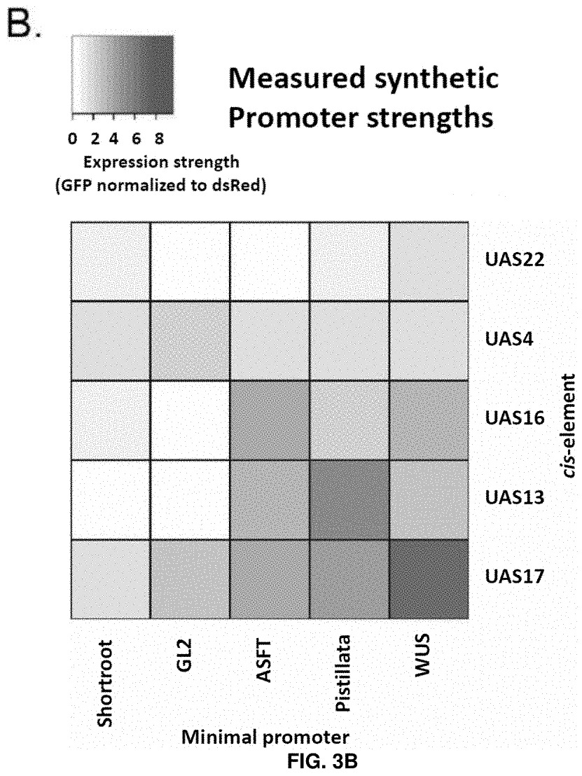

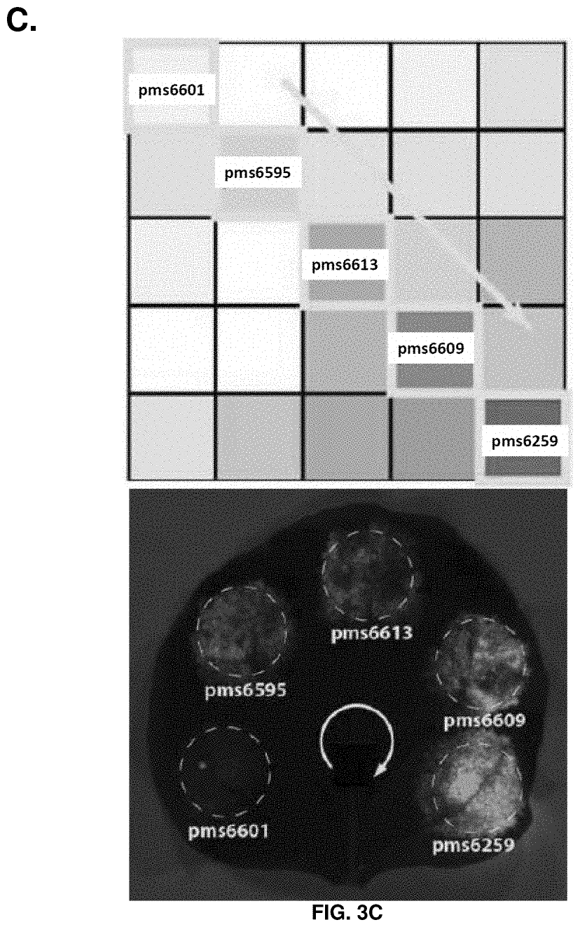

Our library of randomly designed synthetic promoters not only characterizes a distribution of expression strengths, but also provides the opportunity to begin empirically testing the genetic components that make up plant promoter architecture. The comparatively simple and better-characterized promoter structure of prokaryotes has enabled the design of various synthetic promoters^{3, 19}; however, even in the most well studied eukaryotic systems (i.e., yeast), we are only beginning to elucidate the genetic components that dictate gene expression²⁰. To begin testing the contributions of various cis-elements and minimal promoter elements, we constructed a combinatorial library of five random cis-element sequences (each consisting of five concatenated UAS sequences) and five minimal promoters. All twenty-five possible combinations of cis-element and minimal promoter were generated, and the expression strength was measured via GFP fluorescence and normalized to a constitutive promoter driving DsRed expression. The expression profile of all twenty-five promoters reveals an additive effect of promoter elements that contribute to overall expression strength. That is, cis-elements that have a higher affinity for the synthetic activator drive higher gene expression, and similarly, minimal promoters can be identified that increase expression strength as well (FIGS. 3A to 3C). Varying combinations of cis-elements and minimal promoters resulted in an increased range of expression strength (up to 10-fold) versus Gal4 cis-element variation alone (up to 4-fold). The empirical characterization of these synthetic promoters demonstrates the ability to rationally design and engineer synthetic promoters with predicted expression strengths.

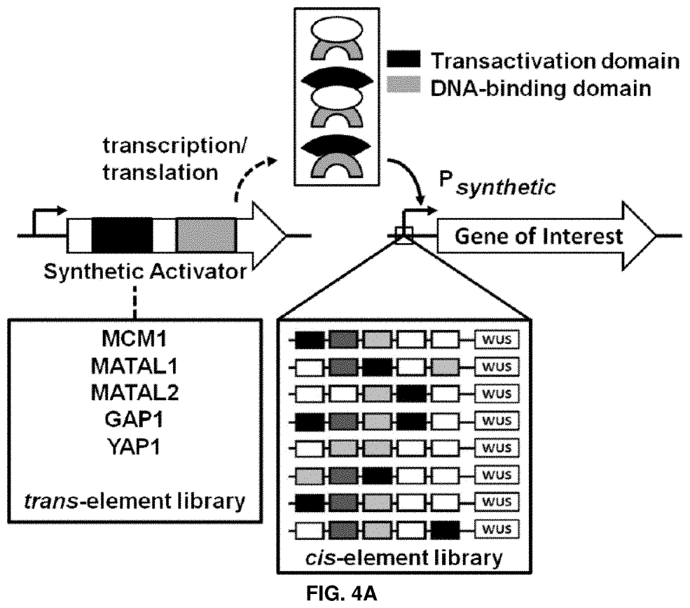

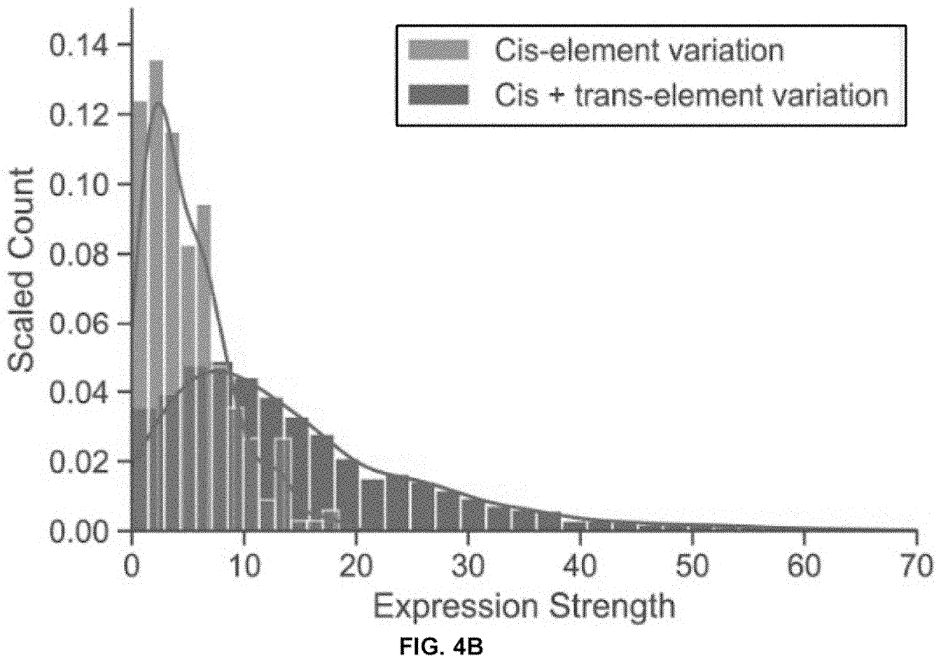

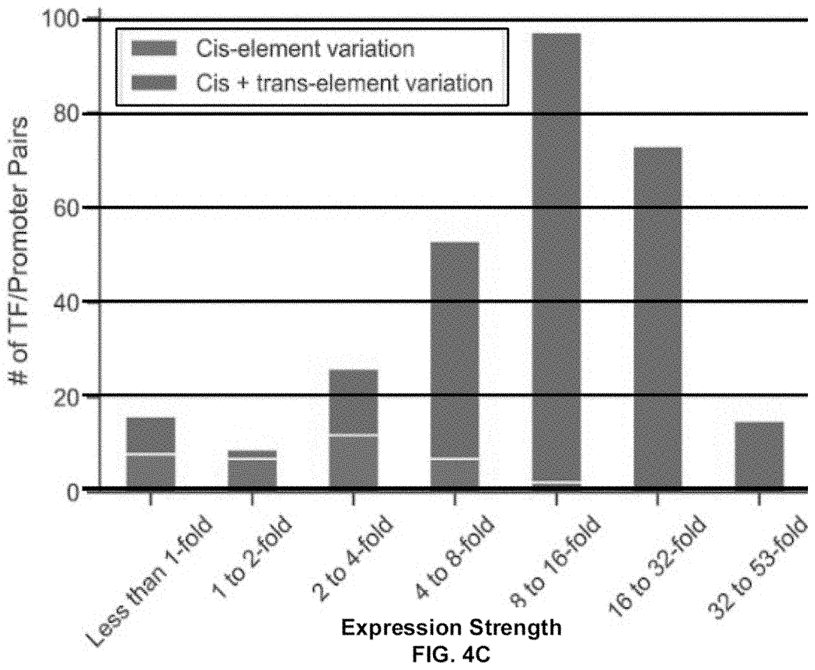

After successfully testing the efficacy of the synthetic Gal4 cis-element system we expanded our approach by examining the effect of alternative and modified trans-elements. We chose five additional, well characterized, transcriptional regulators from S. cerevisiae (YAP1, GAT1, MATAL1, MATAL2, and MCM1) for testing, and designed cis and trans-element libraries for each. We introduced protein modifications, such as fusing an additional transcriptional activator domain to these transcription factors, to further increase the library size and enhance expression strength. We designed additional UASs for the five aforementioned transcription factors using randomly concatenated cis-element motifs and attached them upstream to the plant minimal promoter WUSHCHEL. We tested this cis-element library with the wild-type transcription factor, transcription factor with an additional transcription activating domain fusion, or a minimal DNA-binding scaffold with a transcription activating domain fusion in order to quantify both cis and trans element contributions to expression strength. By expanding our parts library to contain both cis and trans-element variations, we generated an abundance of unique synthetic promoter/TF pairs for plant synthetic biology purposes (FIGS. 4A to 4C). We measured a broader range of expression strengths (up to 50-fold) from our cis/trans-element library than from our cis-element library (up to 10-fold) and our overall library size increased from 25 to over 300 promoter/TF pairs.

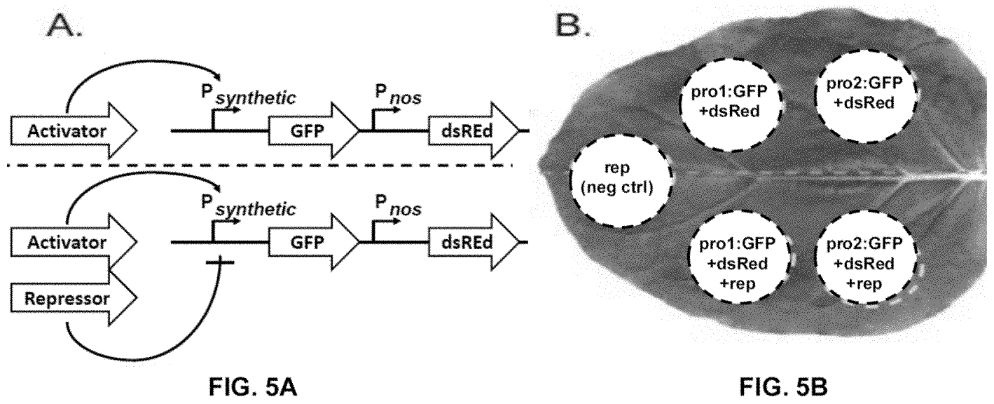

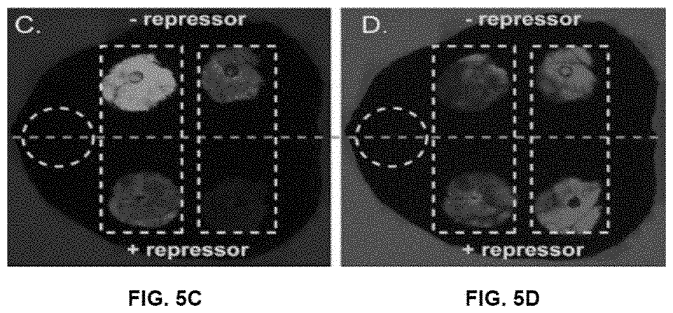

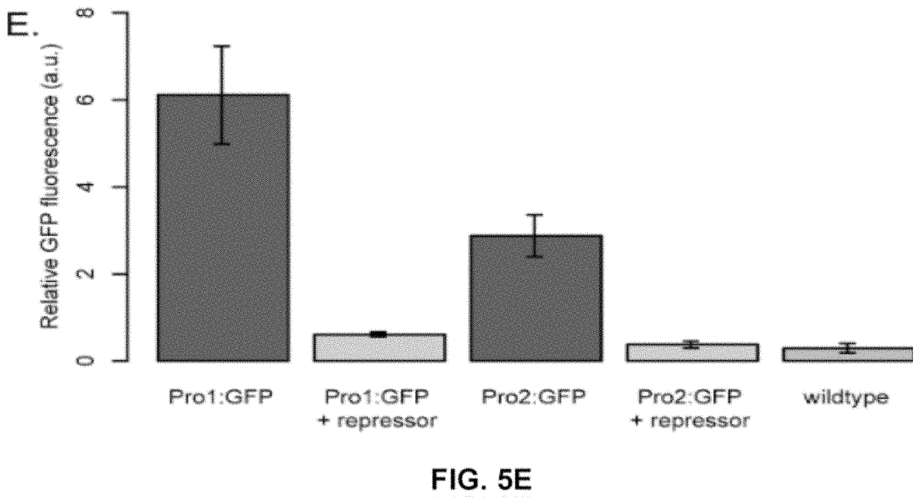

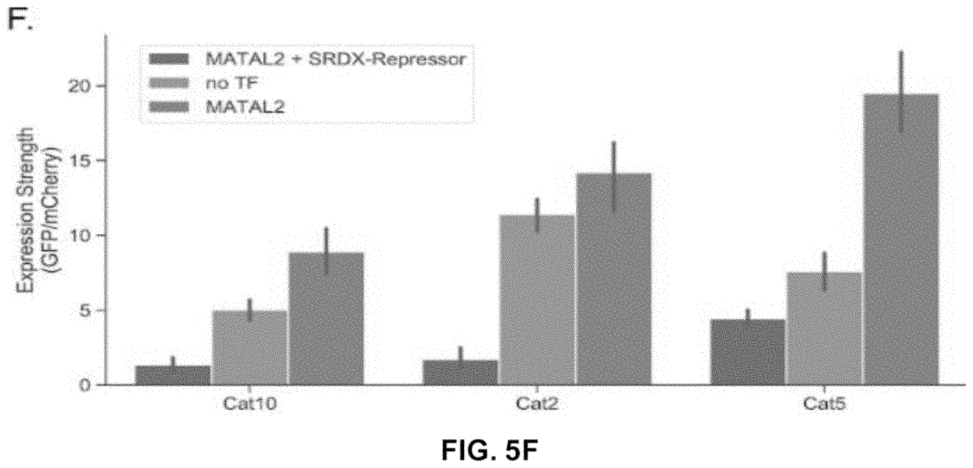

An additional level of control can be designed into our system with the introduction of other regulators, permitting logic to be built into genetic circuits. To explore this possibility, we designed synthetic repressors that bind to our synthetic promoters and repress transcription. As a proof of concept, we fused an SRDX repressor domain to the Gal4 DNA-binding domain¹⁸. When synthetic repressors were co-infiltrated into tobacco leaves with synthetic promoters driving GFP expression, GFP fluorescence decreased, indicative of a repression in gene expression (FIGS. 5A to 5F). Addition of the SRDX repressor to other transcription factor systems also resulted in strong repression of gene expression (FIGS. 5A to 5F). Plant engineering efforts can utilize these synthetic promoters with repressor logic to achieve tissue specific gene repression or build complex gene circuits that can be both activated and repressed.

It has been difficult to unravel the genetic determinants underpinning gene expression in eukaryotes, especially in plants. A core tenet of synthetic biology is the ability to understand the fundamental and reductionist rules that govern natural systems to reconstruct and engineer artificial molecular components of life. Our findings demonstrate the ability to tease apart the additive role of various cis-elements, minimal promoters, and trans-activating elements that dictate gene expression, providing the foundation for future studies to rationally design transcriptional regulation systems with expected expression strengths a priori. Although there are many nuisances to transcriptional regulation in plants that have not been elucidated in this study, our results take one step towards the coarse dissection of the contributory effects of specific genetic elements in controlling gene expression, and future studies may characterize and catalog additional elements that will permit the rational design of custom promoters. These findings may provide the foundation for the future identification of design principles that will enable the construction of more refined and targeted expression of transgenes in plants.

A rapidly growing global population has led to an increase in the motivation and enthusiasm for the engineering of plants that tackle many impending societal challenges. Much of these efforts have focused on agronomy, with the goal of increasing crop output per hectare by optimizing plant traits associated with abiotic/biotic stress, disease resistance, biofortification, and sustainability. Additionally, there has been recent interest in the engineering of crops that produce vitamins and nutrients not native to said crop, with the goal of supplementing the diets of peoples from regions with less access to nutrient rich foods. There is also current interest in the use of plants for “molecular farming”, where engineered plants are grown for the purpose of harvesting therapeutic proteins and high-value compounds. In order to deliver on such agricultural biotechnology solutions, tools will need to be developed that address basic challenges in controlling transgene expression strength, enabling tissue-specific expression, and stacking multiple genes without risks of gene silencing. With these concerns in mind, we have designed a library of synthetic promoters with the intent of addressing a number of these issues and have demonstrated their utility in planta. Improving the capabilities of plant scientists to perform targeted and precise engineering will be indispensable for future complex multi-gene engineering efforts.

### Methods:

Generation of constructs. In order to facilitate the large amount of DNA assembly needed for this study, all constructs described in this study were generated using the yeast assembly-based jStack method, previously described¹⁴. The use of yeast assembly enabled multiple the rapid assembly of various parts into pYB binary vectors, facilitating downstream experiments. Briefly, all the DNA parts were synthesized or cloned as individual DNA parts into starting plasmids (Level 0), with flanking BsaI cut sites. Cassettes composed of linkers, promoters, CDS, and terminator were assembled via Golden Gate cloning²¹, generating Level 1 constructs. Various Level 1 cassettes were linearized and transformed into yeast along with linearized pYB vector in order to facilitate the assembly of all cassettes into the binary vector via homologous stretches of DNA which overlap via Linker and Terminator sequences. All Level 2 constructs were assembled into the binary vector pYB2301¹⁴.

In total, over half a seven hundred thousand base pairs (701,847 bp) of DNA (including Level 0 and Level 1 intermediary shuttle vectors) were synthesized, cloned, and assembled in this study. In total, 461,006 bp of DNA were assembled into binary vectors in this study. All constructs are available through the JBEI ICE registry²².

Design of synthetic promoters. A library of UAS Gal4 binding cis-elements and minimal promoters were collected (Table 1). In order to generate a library of promoters that displayed a broad distribution of expression strengths, five randomly chosen UAS sequences were concatenated together and fused to a random minimal promoter. UAS elements were taken from promoter regions of known genes in the Gal regulon and that have been previously identified based on the seventeen base pair binding motif 5′-CGG-NNNNNNNNNNN-CCG-3′ (SEQ ID NO:100). Known minimal promoter from various plants were synthesized based on previous studies^{11, 12}. DNA promoter parts were synthesized and cloned into the pUC57-Kan vector with flanking BsaI cut sites compatible with standardized Golden Gate²¹ and jStack DNA assembly methods¹⁴.

TABLE 1
Expression strengths of all promoters characterized in initial promoter library described
in FIGS. 2A to 2C. AtAct2 promoter was used to drive the synthetic activator.

| | | | | | Basal |
|---|---|---|---|---|---|
| Promoter | | Construct | Expression | Construct | Expression |
| name | Description | Name | Strength | Name | Strength |
| pms5261 | {P_UAS1:U_HSP70Aly} | pms7526 | 2.12 | pms6018 | 0.19 |
| pms5267 | {P_UAS2:U_PRK} | pms7527 | 4.10 | pms6019 | 0.05 |
| pms5263 | {P_UAS3:U_WUSCHEL} | pms7528 | 4.53 | pms6020 | 0.15 |
| pms5264 | {P_UAS4:U_AT3G24240} | pms7529 | 0.01 | pms6021 | 0.01 |
| pms5930 | {PU_UAS4} | pms5997 | 1.83 | pms6022 | 0.43 |
| pms5931 | {PU_UAS6} | pms5998 | 0.02 | pms6023 | 0.03 |
| pms5932 | {PU_UAS7} | pms5999 | 0.32 | pms6024 | 0.04 |
| pms5933 | {PU_UAS8} | pms6000 | 0.01 | pms6025 | 0.00 |
| pms5934 | {PU_UAS9} | pms6001 | 0.72 | pms6026 | 0.08 |
| pms5936 | {PU_UAS11} | pms6003 | 0.04 | pms6028 | 0.02 |
| pms5937 | {PU_UAS12} | pms6004 | 2.54 | pms6029 | 0.29 |
| pms5938 | {PU_UAS13} | pms6005 | 1.64 | pms6030 | 0.22 |
| pms5939 | {PU_UAS14} | pms6006 | 0.45 | pms6031 | 0.07 |
| pms5940 | {PU_UAS15} | pms6007 | 1.33 | pms6032 | 0.17 |
| pms5941 | {PU_UAS16} | pms6008 | 0.00 | pms6033 | 0.00 |
| pms5942 | {PU_UAS17} | pms6009 | 1.72 | pms6034 | 0.05 |
| pms5943 | {PU_UAS18} | pms6010 | 1.21 | pms6035 | 0.03 |
| pms5944 | {PU_UAS19} | pms6011 | 0.41 | pms6036 | 0.02 |
| pms5945 | {PU_UAS20} | pms6012 | 0.10 | pms6037 | 0.02 |
| pms5946 | {PU_UAS21} | pms6013 | 0.67 | pms6038 | 0.07 |
| pms5948 | {PU_UAS23} | pms6015 | 0.51 | pms6040 | 0.06 |
| pms5949 | {PU_UAS24} | pms6016 | 0.12 | pms6041 | 0.02 |
| pms5950 | {PU_UAS25} | pms6017 | 0.74 | pms6042 | 0.15 |

The synthetic activator was codon-optimized for Arabidopsis and synthesized where the DNA-binding domain of Gal4 was fused to a SV40 NLS and VP16 activator domain on the C-terminus. The synthetic activator was generated by swapping the VP16 activator domain for a SRDX repressor domain.

A second library of thirty-six synthetic promoters were designed and generated (FIGS. 3A to 3C) composed of combinations of various UAS and minimal promoter elements. Six concatenated UAS elements that were generated in the initial synthetic promoter library were chosen along with six minimal promoters. All thirty-six combinations of UAS element fused to a given minimal promoter were synthesized.

Characterization of synthetic promoters. All constructs stacked three basic cassettes: 1) a Gal4 synthetic activator constitutively driven by the Arabidopsis Actin2 promoter, 2) a synthetic promoter driving GFP, and 3) a DsRed constitutively driven by a MAS promoter. All plasmids were assembled into pYB binary vectors using the jStack yeast assembly method¹⁴. Constructs assembled into binary vectors were transformed into Agrobacterium tumefaciens strain GV3101. Transformed Agrobacterium strains were grown in liquid media with appropriate antibiotics and diluted to an OD₆₀₀=1.0. Leaves of four week old N. benthamiana plants were infiltrated following the procedure described in Sparkes et al²³. N. benthamiana plants were grown and maintained in Percival-Scientific growth chambers at 25° C. in 16/8 hour light/dark cycles with 60% humidity. Leaves were collected four days after infiltration, and leaf disks were taken from leaves floated on 200 μL of water in 96 well microtitre plates, and GFP and DsRed fluorescence of each leaf disk was measured using a Synergy 4 microplate reader (Biotek). For each construct eight biological replicates (leaf disks) were taken. Samples were normalized by DsRed expression. Synthetic repressor experiments were measured just as described above, but Agrobacterium strains transformed with binary vectors containing synthetic repressors were co-infiltrated into N. benthamiana leaves.

Arabidopsis transformation. Promoters for the seed-specific (At2S3) and phosphate-inducible promoters (AtPht1.1) were previously cloned from Arabidopsis thaliana Col-0 genomic DNA¹⁴. Cassettes to drive the synthetic activator were built with either the At2S3 or AtPht1.1 promoter upstream of the activator. Reporter genes (GFP, DsRed, and GUS) were driven by synthetic promoters and all Level 1 cassettes were yeast assembled into the binary vector pYB2301 resulting in the final constructs pms5857 and pms6504 respectively, as summarized in Table 2.

TABLE 2
Expression strengths of all promoters characterized in the promoter library rationally
designed to explore the additive effects of various DNA elements described in
FIGS. 4A to 4C. AtAct2 promoter was used to drive the synthetic activator.

| | | | | | Basal |
|---|---|---|---|---|---|
| Promoter | | Construct | Expression | Construct | Expression |
| name | Description | Name | Strength | Name | Strength |
| pms6189 | {P_UAS17:WUSHCEL} | pms6259 | 9.64 | pms6370 | 1.24 |
| pms6188 | {P_UAS16:WUSHCEL} | pms6258 | 3.25 | pms6369 | 0.26 |
| pms6185 | {P_UAS13:WUSHCEL} | pms6255 | 2.92 | pms6366 | 0.36 |
| pms6176 | {P_UAS4:WUSHCEL} | pms6246 | 1.66 | pms6357 | 0.08 |
| pms6194 | {P_UAS22:WUSHCEL} | pms6264 | 1.50 | pms6375 | 0.63 |
| pms6542 | {P_UAS17:GL2} | pms6592 | 2.65 | pms6642 | 0.02 |
| pms6543 | {P_UAS16:GL2} | pms6593 | 0.30 | pms6643 | 0.01 |
| pms6544 | {P_UAS13:GL2} | pms6594 | 0.32 | pms6644 | 0.02 |
| pms6545 | {P_UAS4:GL2} | pms6595 | 2.26 | pms6645 | 0.01 |
| pms6546 | {P_UAS22:GL2} | pms6596 | 0.13 | pms6646 | 0.01 |
| pms6547 | {P_UAS17:Shortroot} | pms6597 | 1.40 | pms6647 | 0.03 |
| pms6548 | {P_UAS16:Shortroot} | pms6598 | 0.97 | pms6648 | 0.03 |
| pms6549 | {P_UAS13:Shortroot} | pms6599 | 0.00 | pms6649 | 0.06 |
| pms6550 | {P_UAS4:Shortroot} | pms6600 | 1.39 | pms6650 | 0.02 |
| pms6551 | {P_UAS22:Shortroot} | pms6601 | 0.81 | pms6651 | 0.04 |
| pms6557 | {P_UAS17:Pistillata} | pms6607 | 5.14 | pms6657 | 0.10 |
| pms6558 | {P_UAS16:Pistillata} | pms6608 | 2.24 | pms6658 | 0.12 |
| pms6559 | {P_UAS13:Pistillata} | pms6609 | 6.11 | pms6659 | 0.08 |
| pms6560 | {P_UAS4:Pistillata} | pms6610 | 1.34 | pms6660 | 0.11 |
| pms6561 | {P_UAS22:Pistillata} | pms6611 | 1.14 | pms6661 | 0.04 |
| pms6562 | {P_UAS17:ASFT} | pms6612 | 4.26 | pms6662 | 0.04 |
| pms6563 | {P_UAS16:ASFT} | pms6613 | 4.26 | pms6663 | 0.08 |
| pms6564 | {P_UAS13:ASFT} | pms6614 | 3.79 | pms6664 | 0.05 |
| pms6565 | {P_UAS4:ASFT} | pms6615 | 1.29 | pms6665 | 0.03 |
| pms6566 | {P_UAS22:ASFT} | pms6616 | 0.00 | pms6666 | 0.04 |

The plasmids were each transformed into the Agrobacterium tumefaciens strain GV3101, which was subsequently used for transformation into Arabidopsis Col-0 background using the floral dip infiltration method²⁴. Transformed Arabidopsis plants were selected by plating the resulting T1 seeds onto agar plates containing ½ Murashige and Skoog (Phytotechlab, webpage for: phytotechlab.com/), 1% (w/v) sucrose, and 50 μg mL-1 Kanamycin. After 2 weeks, resistant plants were moved to soil.

Plant growth conditions. Arabidopsis seeds were surface sterilized, rinsed with sterile water and stratified at 4° C. for 3 days. Seeds were germinated on agar plates with half-strength MS (½ MS) salts containing 1% sucrose with kanamycin resistance for 2 weeks. Seedlings were grown vertically on agar plates in growth chamber at 21-23° C. for 2 weeks with a light intensity of 100-130 μE m⁻²s⁻ under a short-day light cycle (10 h light/14 h dark).***

### Phosphate Availability Experiments.

For the phosphate experiments, seeds were grown under sterile conditions on vertical agar plates with one set containing half-strength MS (½ MS) salts lacking phosphate, iron and nitrogen (Phytotechlab, webpage for: phytotechlab.com/) supplemented with nitrogen (5 mM ammonium nitrate) and iron (100 μM Fe-EDTA), to only lack phosphate content. Where seedlings were grown on no phosphate, the KH₂PO₄/K₂HPO₄was replaced with KCl to maintain the potassium ion concentration in the medium. The other set were supplemented with all the above mentioned nutrients as well as 2 mM phosphate (KH₂PO₄) all containing 1% (w/v) sucrose with kanamycin resistance for 2 weeks.

For the phosphate experiments, the seeds were grown on ½ MS media agar plates (Phytotechlab, webpage for: phytotechlab.com/) with (2 mM KH₂PO₄) and without phosphate with 1% (w/v) sucrose with kanamycin resistance for 2 weeks. Where seedlings were grown on no phosphate, the KH₂PO₄/K₂HPO₄was replaced with KCl to maintain the potassium ion concentration in the medium.

### Microscope Analysis.

A laser scanning confocal microscope (LSM 710; Carl Zeiss Microscopy) was used for fluorescence analysis of Arabidopsis plants stably transformed with the reporter genes. Excitation of GFP and DsRed was performed using lasers at 488 with emission filter 510-530 nm and 558 nm with emission filter 583-592 nm, respectively 2 week old seedlings expressing GFP and DsRed were used for imaging.

### Histochemical GUS Staining.

To assay β-glucuronidase (GUS) reporter activity, whole DsRED- or GFP-positive seeds and seedlings were infiltrated with staining solution (1 mM EDTA, 0.2% Triton X-100, 0.2% Tween-20 in TBS, pH 7.3) containing 1 mM 5-bromo-4-chloro-3-indolyl-β-D-glucuronide (X-Gluc). Ferricyanide (0.25 mM) was added to prevent indigo precursor migration25. The chelator EDTA was added to the staining solution to prevent any gene expression during the staining procedure. In seedlings, substrate penetration was assisted by two vacuum infiltrations at 0.1 atm for 15 min each on ice to improve infiltration. In seeds, substrate penetration was assisted by incubating around 20 seeds in round filter papers, moistened with water and placed in a plastic petri dish. After 3-day pre-chilling at 4° C. and 22h incubation at 22° C., the small filter papers supporting the seeds were briefly blotted on dry filter papers to remove excessive water and subsequently GUS staining was carried out²⁶. The seedlings and seeds were incubated in staining solution at 37° C. until sufficient blue staining had been developed.

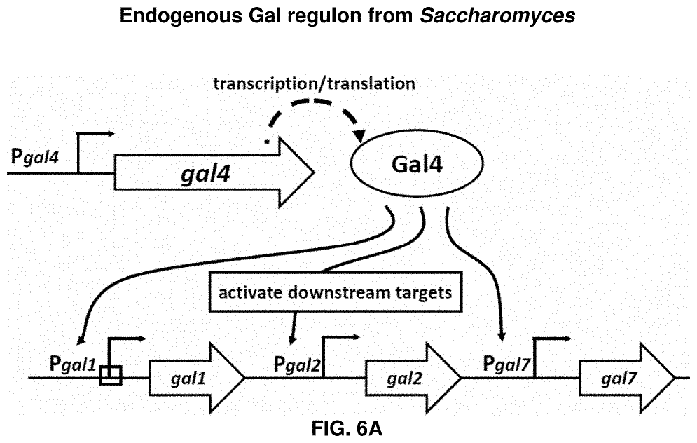

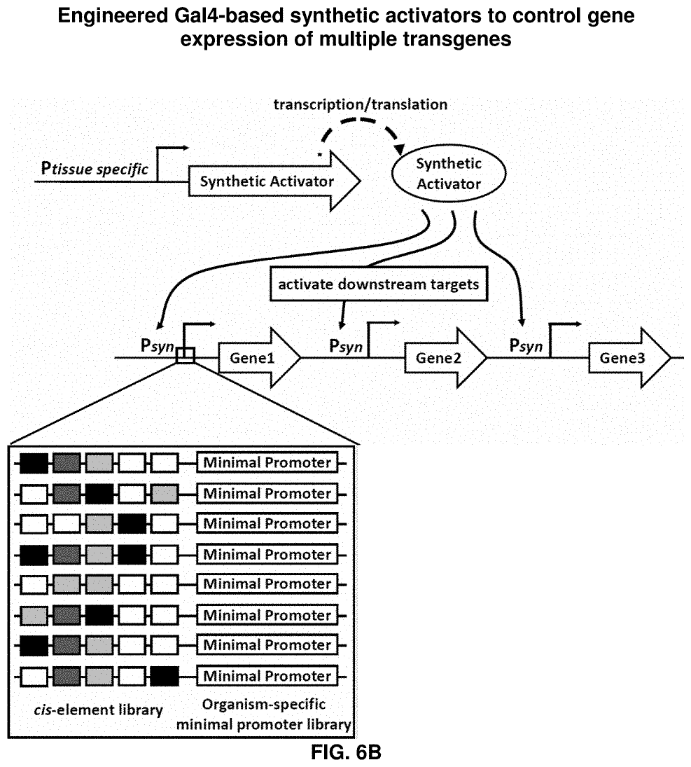

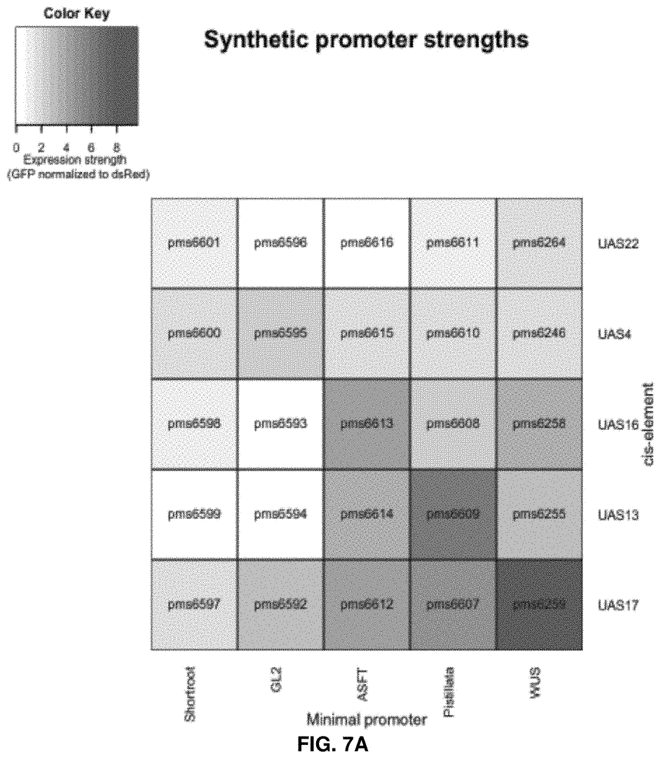

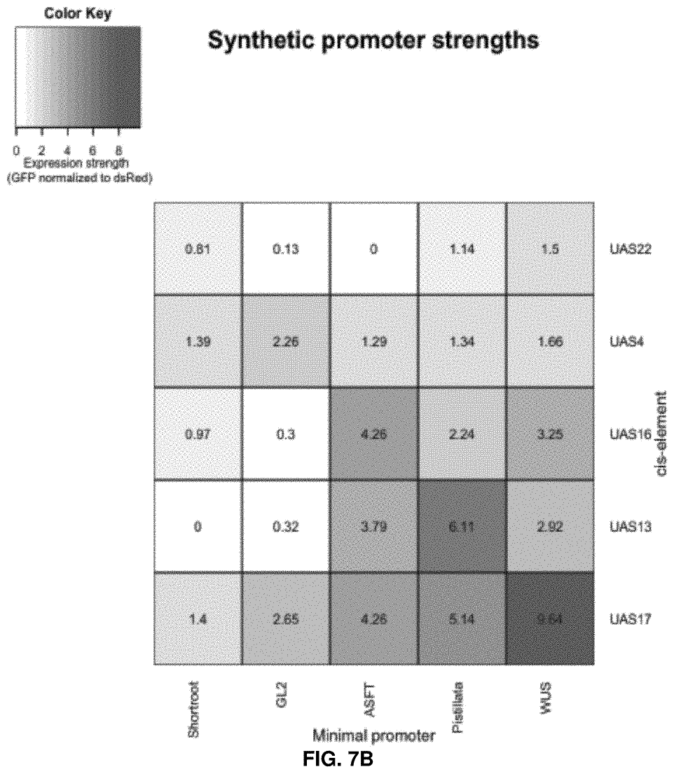

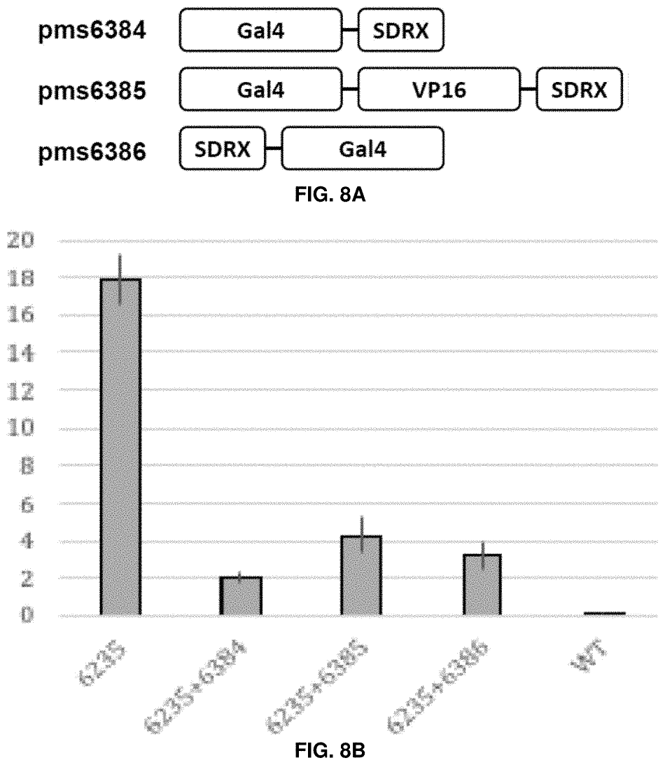

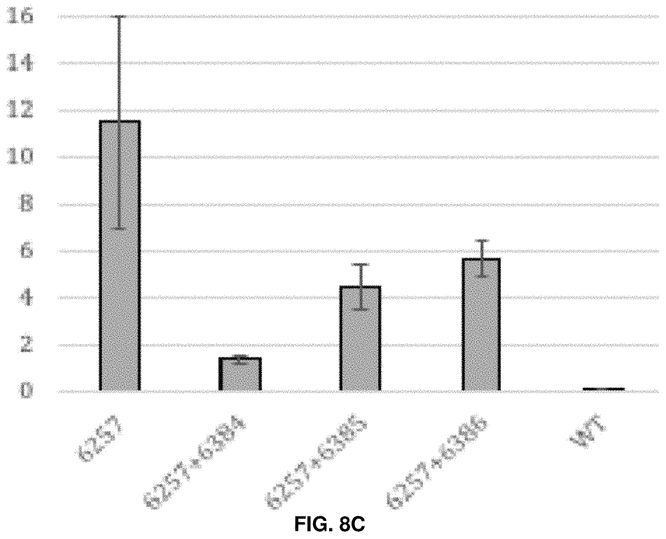

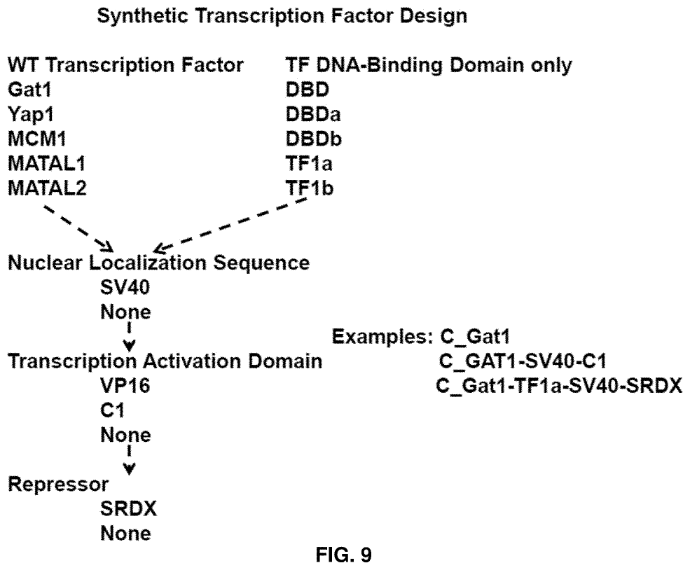

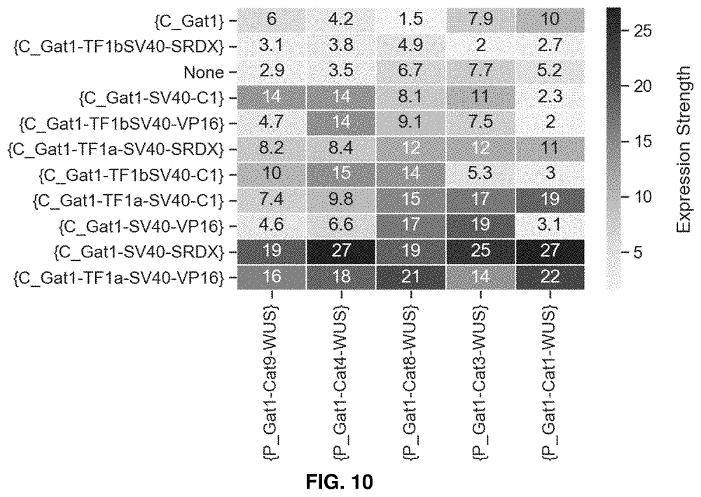

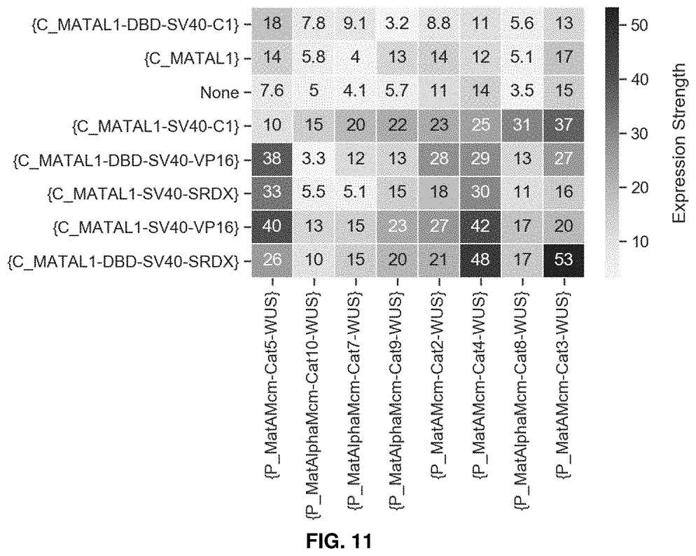

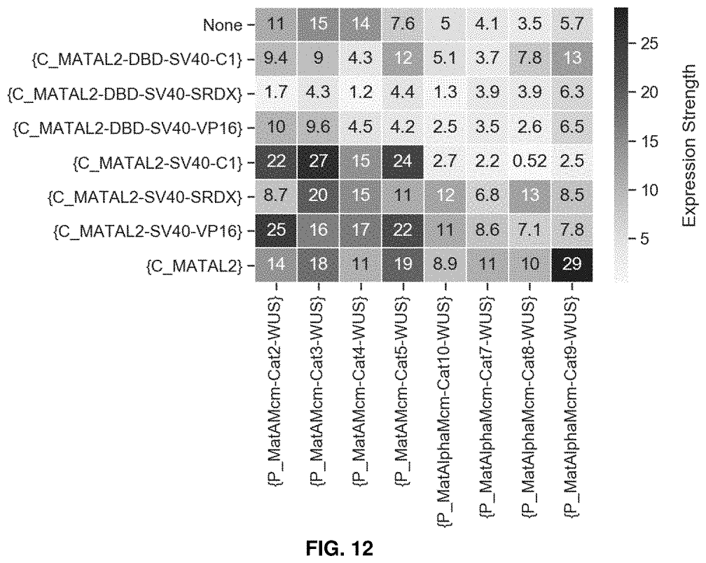

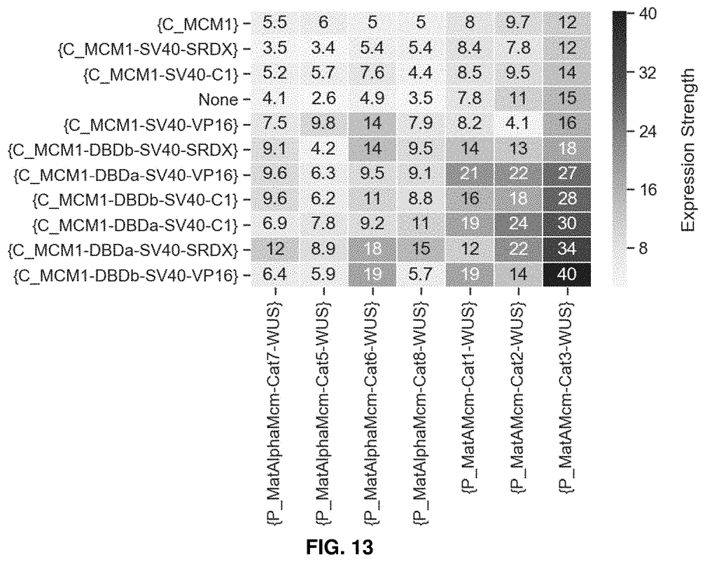

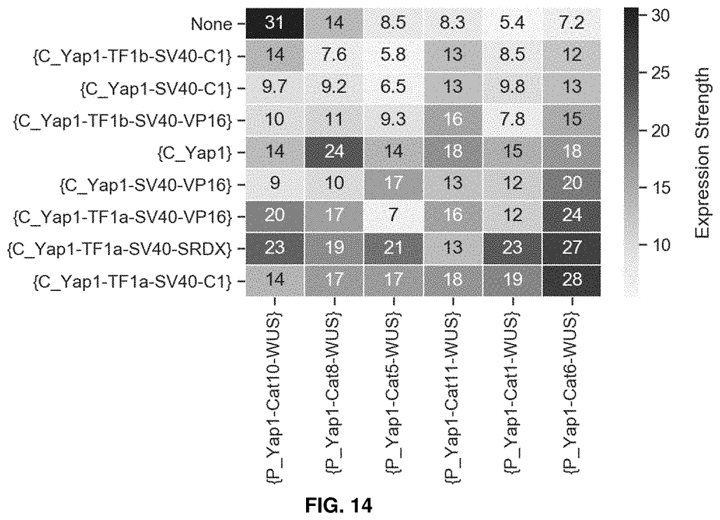

Further embodiments of the invention, and data showing the making and using of the invention, are shown in FIG. 6A to 14.

### REFERENCES CITED

1 Shih, P. M., Liang, Y. & Logué, D. Biotechnology and synthetic biology approaches for metabolic engineering of bioenergy crops. Plant J 87, 103-117 (2016).
2 Salis, H. M., Mirsky, E. A. & Voigt, C. A. Automated design of synthetic ribosome binding sites to control protein expression. Nat Biotech 27, 946-950 (2009).
3 Mutalik, V. K. et al. Precise and reliable gene expression via standard transcription and translation initiation elements. Nat Meth 10, 354-360 (2013).
4 Hawkins, K. M. & Smolke, C. D. Production of benzylisoquinoline alkaloids in Saccharomyces cerevisiae. Nat Chem Biol 4, 564-573 (2008).
5 Potvin-Trottier, L., Lord, N. D., Vinnicombe, G. & Paulsson, J. Synchronous long-term oscillations in a synthetic gene circuit. Nature 538, 514-517 (2016).
6 Sunilkumar, G., Mohr, L., Lopata-Finch, E., Emani, C. & Rathore, K. S. Developmental and tissue-specific expression of CaMV 35S promoter in cotton as revealed by GFP. Plant Mol Biol 50, 463-479 (2002).
7 Liu, W. et al. Computational discovery of soybean promoter cis-regulatory elements for the construction of soybean cyst nematode-inducible synthetic promoters. Plant Biotechnology Journal 12, 1015-1026, doi:10.1111/pbi.12206 (2014).
8 Fagard, M. & Vaucheret, H. (Trans)Gene silencing in plants: How Many Mechanisms? Annu Rev Plant Physiol Plant Mol Biol 51, 167-194 (2000).
9 Matzke, M. A., Primig, M., Trnovsky, J. & Matzke, A. J. M. Reversible methylation and inactivation of marker genes in sequentially transformed tobacco plants. EMBO J 8, 643-649 (1989).
10 Sadowski, I., Ma, J., Triezenberg, S. & Ptashne, M. GAL4-VP16 is an unusually potent transcriptional activator. Nature 335, 563-564 (1988).
11 Kiran, K. et al. The TATA-Box Sequence in the Basal Promoter Contributes to Determining Light-Dependent Gene Expression in Plants. Plant Physiol 142, 364-376 (2006).
12 Joshi, C. P. An inspection of the domain between putative TATA box and translation start site in 79 plant genes. Nucleic Acids Research 15, 6643-6653 (1987).
13 Lubliner, S. et al. Core promoter sequence in yeast is a major determinant of expression level. Genome Res 25, 1008-1017 (2015).
14 Shih, P. M. et al. A robust gene-stacking method utilizing yeast assembly for plant synthetic biology. Nature Comm 7, 13215 (2016).
15 Harcum, S. W. & Bentley, W. E. Heat-shock and stringent responses have overlapping protease activity in Escherichia coli. Implications for heterologous protein yield. Appl Biochem Biotechnol 80, 23-37 (1999).
16 Tian, D., Traw, M. B., Chen, J. Q., Kreitman, M. & Bergelson, J. Fitness costs of R-gene-mediated resistance in Arabidopsis thaliana. Nature 423, 74-77 (2003).
17 Denancé, N., Sánchez-Vallet, A., Goffner, D. & Molina, A. Disease resistance or growth: the role of plant hormones in balancing immune responses and fitness costs. Front Plant Sci 4, 155 (2013).
18 Hiratsu, K., Matsui, K., Koyama, T. & Ohme-Takagi, M. Dominant repression of target genes by chimeric repressors that include the EAR motif, a repression domain, in Arabidopsis. Plant J34, 733-739 (2003).
19 Jensen, P. R. & Hammer, K. The Sequence of Spacers between the Consensus Sequences Modulates the Strength of Prokaryotic Promoters. Appl Environ Microbiol 64, 82-87 (1998).
20 Levo, M. & Segal, E. In pursuit of design principles of regulatory sequences. Nat Rev Genet 15, 453-468 (2014).
21 Patron, N. J. et al. Standards for plant synthetic biology: a common syntax for exchange of DNA parts. New Phytol 208, 13-19, doi:10.1111/nph.13532 (2015).
22 Ham, T. S. et al. Design, implementation and practice of JBEI-ICE: an open source biological part registry platform and tools. Nucleic Acids Res 40, e141-e141 (2012).
23 Sparkes, I. A., Runions, J., Kearns, A. & Hawes, C. Rapid, transient expression of fluorescent fusion proteins in tobacco plants and generation of stably transformed plants. Nat Protocols 1, 2019-2025 (2006).
24 Clough, S. J. & Bent, A. F. Floral dip: a simplified method for Agrobacterium-mediated transformation of Arabidopsis thaliana. Plant J 16, 735-743 (1998).
25 Jefferson, R. A. Assaying chimeric genes in plants: The GUS gene fusion system. Plant Mol Biol Reporter 5, 387-405 (1997).
26 Liu, P.-P., Koizuka, N., Martin, R. C. & Nonogaki, H. The BME3 (Blue Micropylar End 3) GATA zinc finger transcription factor is a positive regulator of Arabidopsis seed germination. Plant J 44, 960-971 (2005).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A synthetic transcription factor (TF) comprising (a) a DNA-binding domain of a transcription factor linked to (b) an activator domain or repressor domain, and (c) a nuclear localization sequence (NLS).

2. The synthetic TF of claim 1, wherein the eukaryotic TF is a Saccharomyces cerevisiae TF.

3. The synthetic TF of claim 2, wherein the S. cerevisiae TF is Gal4, YAP1, GAT1, MATAL1, MATAL2, MCM1, Abf1, Adr1, Ash1, Gcn4, Gcr1, Hap4, Hsf1, Ime1, Ino2/Ino4, Leu3, Lys14, Matα2, Mga2, Met4, Mig1, Rap1, Rgt1, Rlm1, Smp1, Rme1, Rox1, Rtg3, Spt23, Tea1, Ume6, or Zap1.

4. The synthetic TF of claim 3, wherein the S. cerevisiae TG is Gal4, YAP1, GAT1, MATAL1, MATAL2, or MCM1.

5. The synthetic TF of claim 1, wherein the synthetic TF comprises the activator domain which is a herpes simplex virus VP16, maize C1, or a yeast activator domain.

6. The synthetic TF of claim 5, wherein the S. cerevisiae activator domain is a Rap1 activator domain.

7. The synthetic TF of claim 1, wherein the DNA-binding domain comprises the amino acid sequence of Rap1, or GXXIRXRF (wherein X is any amino acid), G(G, P, A or R)(S or A)IRXRF (wherein X is any amino acid), or GNSIRHRFRV.

8. The synthetic TF of claim 1, wherein the synthetic TF comprises the repressor domain.

9. The synthetic TF of claim 8 wherein the repressor domain comprises an EAR motif, TLLLFR motif, R/KLFGV motif. LxLxPP motif, or a yeast repressor domain.

10. The synthetic TF of claim 9, herein the yeast repressor domain is a Saccharomyces cerevisiae Ash1, Matα2, Mig1, Rap1, Rgt1, Rme1, Rox1, or Urne6 repressor domain.

11. The synthetic TF of claim 1, wherein the NLS comprises a M9 domain or PY-NLS motif.

12. The synthetic TF of claim 1, wherein the NLS comprises the amino acid sequence KIPIK (yeast Matα2).

13. The synthetic TF of claim 1 wherein any two, or all, of the DNA-binding domain, the activator domain, the repressor domain, and the NLS are heterologous to each other.

14. The synthetic TF of claim 1, wherein one or more, or all, of the DNA-binding domain, the activator domain, the repressor domain, and the NLS are obtained or derived from a. non-viral organism.

15. The synthetic TF of claim 1, wherein the DNA-binding domain, the NLS, and the activator domain or repressor domain are linked in this order from N- to C-terminus.

16. A nucleic acid encoding the synthetic TF of claim 1 operatively linked to a promoter capable of expressing the synthetic TF in vitro or in vivo.

17. A vector comprising the nucleic acid of claim 16.

18. A host cell comprising the vector of claim 17, wherein the host cell is capable of expressing the synthetic TF.

19. A system comprising a nucleic acid of claim 16 and a second nucleic acid, or the nucleic acid, encodes a gene of interest (GOI) operatively linked to a promoter and one or more activator/repressor binding domains, or combination thereof, wherein the synthetic TF binds at least one of the one or more activator/repressor binding domain such that the synthetic TF modulates the expression of the GOI.

20. A genetically modified eukaryotic cell or organism comprising: (a) (i) one or more nucleic acids each encoding one or more transcription activators operatively linked to a first promoter, (ii) one or more nucleic acids each encoding one or more transcription repressors each operatively linked to a second promoter, or (iii) combinations thereof; and (b) one or more nucleic acids each encoding one or more independent genes of interest (GOI) each operatively linked to a promoter that is activated by the one or more transcription activators, repressed by the one or more transcription repressors, or a combination of both; wherein at least one transcription activator or transcription repressor is a synthetic transcription factor (TF) of claim 1.


SEQUENCE LISTING (104 sequences)

SEQ ID NO: 1 - protein, 372 aa, Artificial Sequence
  Description of Artificial Sequence Synthetic polypeptide
  Synthetic fusion protein MCM1 SV40 VP16 domains
MSDIEEGTPTNNGQQKERRKIEIKFIENKTRRHVTFSKRKHGIMKKAFELSVLTGTQVLLLVVSETGLVYTFSTPKFEPIVTQQEGRNLIQACLNAPDDEEEDEEEDGDDDDDDDDDGNDMQRQQPQQQQPQQQQQVLNAHANSLGHLNQDQVPAGALKQEVKSQLLGGANPNQNSMIQQQQHHTQNSQPQQQQQQQPQQQMSQQQMSQHPRPQQGIPHPQQSQPQQQQQQQQQLQQQQQQQQQQPLTGIHQPHQQAFANAASPYLNAEQNAAYQQYFQEPQQGQYGPKKKRKVAPPTDVSLGDELHLDGEDVAMAHADALDDFDLDMLGDGDSPGPGFTPHDSAPYGALDMADFEFEQMFTDALGIDEYGG

SEQ ID NO: 2 - protein, 142 aa, Artificial Sequence
  Description of Artificial Sequence Synthetic polypeptide
  Synthetic fusion protein MCM1 DBDa SV40 VP16 domains
MRRKIEIKFIENKTRRHVTFSKRKHGIMKKAFELSVLTGTQVLLLVVSETGLVYTFGPKKKRKVAPPTDVSLGDELHLDGEDVAMAHADALDDFDLDMLGDGDSPGPGFTPHDSAPYGALDMADFEFEQMFTDALGIDEYGG

SEQ ID NO: 3 - protein, 168 aa, Artificial Sequence
  Description of Artificial Sequence Synthetic polypeptide
  Synthetic fusion protein MCM1 DBDb SV40 VP16 domains
MKERRKIEIKFIENKTRRHVTFSKRKHGIMKKAFELSVLTGTQVLLLVVSETGLVYTFSTPKFEPIVTQQEGRNLIQACLNAGPKKKRKVAPPTDVSLGDELHLDGEDVAMAHADALDDFDLDMLGDGDSPGPGFTPHDSAPYGALDMADFEFEQMFTDALGIDEYGG

SEQ ID NO: 4 - protein, 418 aa, Artificial Sequence
  Description of Artificial Sequence Synthetic polypeptide
  Synthetic fusion protein MCM1 SV40 C1 domains
MSDIEEGTPTNNGQQKERRKIEIKFIENKTRRHVTFSKRKHGIMKKAFELSVLTGTQVLLLVVSETGLVYTFSTPKFEPIVTQQEGRNLIQACLNAPDDEEEDEEEDGDDDDDDDDDGNDMQRQQPQQQQPQQQQQVLNAHANSLGHLNQDQVPAGALKQEVKSQLLGGANPNQNSMIQQQQHHTQNSQPQQQQQQQPQQQMSQQQMSQHPRPQQGIPHPQQSQPQQQQQQQQQLQQQQQQQQQQPLTGIHQPHQQAFANAASPYLNAEQNAAYQQYFQEPQQGQYGPKKKRKVCETGQNSAAHRADPDSAGTTTTSAAAVWAPKAVRCTGGLFFFHRDTTPAHAGETATPMAGGGGGGGGEAGSSDDCSSAASVSLRVGSHDEPCFSGDGDGDWMDDVRALASFLESDEDWLRCQTA

SEQ ID NO: 5 - protein, 188 aa, Artificial Sequence
  Description of Artificial Sequence Synthetic polypeptide
  Synthetic fusion protein MCM1 DBDa SV40 C1 domains
MRRKIEIKFIENKTRRHVTFSKRKHGIMKKAFELSVLTGTQVLLLVVSETGLVYTFGPKKKRKVCETGQNSAAHRADPDSAGTTTTSAAAVWAPKAVRCTGGLFFFHRDTTPAHAGETATPMAGGGGGGGGEAGSSDDCSSAASVSLRVGSHDEPCFSGDGDGDWMDDVRALASFLESDEDWLRCQTA

SEQ ID NO: 6 - protein, 214 aa, Artificial Sequence
  Description of Artificial Sequence Synthetic polypeptide
  Synthetic fusion protein MCM1 DBDb SV40 C1 domains
MKERRKIEIKFIENKTRRHVTFSKRKHGIMKKAFELSVLTGTQVLLLVVSETGLVYTFSTPKFEPIVTQQEGRNLIQACLNAGPKKKRKVCETGQNSAAHRADPDSAGTTTTSAAAVWAPKAVRCTGGLFFFHRDTTPAHAGETATPMAGGGGGGGGEAGSSDDCSSAASVSLRVGSHDEPCFSGDGDGDWMDDVRALASFLESDEDWLRCQTA

SEQ ID NO: 7 - protein, 212 aa, Artificial Sequence
  Description of Artificial Sequence Synthetic polypeptide
  Synthetic fusion protein MATAL1 SV40 VP16 domains
MDDICSMAENINRTLFNILGTEIDEINLNTNNLYNFIMESNLTKVEQHTLHKNISNNRLEIYHHIKKEKSPKGKSSISPQARAFLEQVFRRKQSLNSKEKEEVAKKCGITPLQVRVWFINKRMRSKGPKKKRKVAPPTDVSLGDELHLDGEDVAMAHADALDDFDLDMLGDGDSPGPGFTPHDSAPYGALDMADFEFEQMFTDALGIDEYGG

SEQ ID NO: 8 - protein, 148 aa, Artificial Sequence
  Description of Artificial Sequence Synthetic polypeptide
  Synthetic fusion protein MATAL1 DBD SV40 VP16 domains
MKKEKSPKGKSSISPQARAFLEQVFRRKQSLNSKEKEEVAKKCGITPLQVRVWFINKRMRSKGPKKKRKVAPPTDVSLGDELHLDGEDVAMAHADALDDFDLDMLGDGDSPGPGFTPHDSAPYGALDMADFEFEQMFTDALGIDEYGG

SEQ ID NO: 9 - protein, 258 aa, Artificial Sequence
  Description of Artificial Sequence Synthetic polypeptide
  Synthetic fusion protein MATAL1 SV40 C1 domains
MDDICSMAENINRTLFNILGTEIDEINLNTNNLYNFIMESNLTKVEQHTLHKNISNNRLEIYHHIKKEKSPKGKSSISPQARAFLEQVFRRKQSLNSKEKEEVAKKCGITPLQVRVWFINKRMRSKGPKKKRKVCETGQNSAAHRADPDSAGTTTTSAAAVWAPKAVRCTGGLFFFHRDTTPAHAGETATPMAGGGGGGGGEAGSSDDCSSAASVSLRVGSHDEPCFSGDGDGDWMDDVRALASFLESDEDWLRCQTA

SEQ ID NO: 10 - protein, 194 aa, Artificial Sequence
  Description of Artificial Sequence Synthetic polypeptide
  Synthetic fusion protein MATAL1 DBD SV40 C1 domains
MKKEKSPKGKSSISPQARAFLEQVFRRKQSLNSKEKEEVAKKCGITPLQVRVWFINKRMRSKGPKKKRKVCETGQNSAAHRADPDSAGTTTTSAAAVWAPKAVRCTGGLFFFHRDTTPAHAGETATPMAGGGGGGGGEAGSSDDCSSAASVSLRVGSHDEPCFSGDGDGDWMDDVRALASFLESDEDWLRCQTA

SEQ ID NO: 11 - protein, 296 aa, Artificial Sequence
  Description of Artificial Sequence Synthetic polypeptide
  Synthetic fusion protein MATAL2 SV40 VP16 domains
MNKIPIKDLLNPQITDEFKSSILDINKKLFSICCNLPKLPESVTTEEEVELRDILGFLSRANKNRKISDEEKKLLQTTSQLTTTITVLLKEMRSIENDRSNYQLTQKNKSADGLVFNVVTQDMINKSTKPYRGHRFTKENVRILESWFAKNIENPYLDTKGLENLMKNTSLSRIQIKNWVSNRRRKEKTITIAPELADLLSGEPLAKKKEGPKKKRKVAPPTDVSLGDELHLDGEDVAMAHADALDDFDLDMLGDGDSPGPGFTPHDSAPYGALDMADFEFEQMFTDALGIDEYGG

SEQ ID NO: 12 - protein, 155 aa, Artificial Sequence
  Description of Artificial Sequence Synthetic polypeptide
  Synthetic fusion protein MATAL2 DBD SV40 VP16 domains
MSTKPYRGHRFTKENVRILESWFAKNIENPYLDTKGLENLMKNTSLSRIQIKNWVSNRRRKEKTITIAPGPKKKRKVAPPTDVSLGDELHLDGEDVAMAHADALDDFDLDMLGDGDSPGPGFTPHDSAPYGALDMADFEFEQMFTDALGIDEYGG

SEQ ID NO: 13 - protein, 342 aa, Artificial Sequence
  Description of Artificial Sequence Synthetic polypeptide
  Synthetic fusion protein MATAL2 SV40 C1 domains
MNKIPIKDLLNPQITDEFKSSILDINKKLFSICCNLPKLPESVTTEEEVELRDILGFLSRANKNRKISDEEKKLLQTTSQLTTTITVLLKEMRSIENDRSNYQLTQKNKSADGLVFNVVTQDMINKSTKPYRGHRFTKENVRILESWFAKNIENPYLDTKGLENLMKNTSLSRIQIKNWVSNRRRKEKTITIAPELADLLSGEPLAKKKEGPKKKRKVCETGQNSAAHRADPDSAGTTTTSAAAVWAPKAVRCTGGLFFFHRDTTPAHAGETATPMAGGGGGGGGEAGSSDDCSSAASVSLRVGSHDEPCFSGDGDGDWMDDVRALASFLESDEDWLRCQTA

SEQ ID NO: 14 - protein, 201 aa, Artificial Sequence
  Description of Artificial Sequence Synthetic polypeptide
  Synthetic fusion protein  MATAL2 DBD SV40 C1 domains
MSTKPYRGHRFTKENVRILESWFAKNIENPYLDTKGLENLMKNTSLSRIQIKNWVSNRRRKEKTITIAPGPKKKRKVCETGQNSAAHRADPDSAGTTTTSAAAVWAPKAVRCTGGLFFFHRDTTPAHAGETATPMAGGGGGGGGEAGSSDDCSSAASVSLRVGSHDEPCFSGDGDGDWMDDVRALASFLESDEDWLRCQTA

SEQ ID NO: 15 - protein, 736 aa, Artificial Sequence
  Description of Artificial Sequence Synthetic polypeptide
  Synthetic fusion protein Yap1 SV40 VP16 domains
MSVSTAKRSLDVVSPGSLAEFEGSKSRHDEIENEHRRTGTRDGEDSEQPKKKGSKTSKKQDLDPETKQKRTAQNRAAQRAFRERKERKMKELEKKVQSLESIQQQNEVEATFLRDQLITLVNELKKYRPETRNDSKVLEYLARRDPNLHFSKNNVNHSNSEPIDTPNDDIQENVKQKMNFTFQYPLDNDNDNDNSKNVGKQLPSPNDPSHSAPMPINQTQKKLSDATDSSSATLDSLSNSNDVLNNTPNSSTSMDWLDNVIYTNRFVSGDDGSNSKTKNLDSNMFSNDFNFENQFDEQVSEFCSKMNQVCGTRQCPIPKKPISALDKEVFASSSILSSNSPALTNTWESHSNITDNTPANVIATDATKYENSFSGFGRLGFDMSANHYVVNDNSTGSTDSTGSTGNKNKKNNNNSDDVLPFISESPFDMNQVTNFFSPGSTGIGNNAASNTNPSLLQSSKEDIPFINANLAFPDDNSTNIQLQPFSESQSQNKFDYDMFFRDSSKEGNNLFGEFLEDDDDDKKAANMSDDESSLIKNQLINEEPELPKQYLQSVPGNESEISQKNGSSLQNADKINNGNDNDNDNDVVPSKEGSLLRCSEIWDRITTHPKYSDIDVDGLCSELMAKAKCSERGVVINAEDVQLALNKHMNGPKKKRKVAPPTDVSLGDELHLDGEDVAMAHADALDDFDLDMLGDGDSPGPGFTPHDSAPYGALDMADFEFEQMFTDALGIDEYGG

SEQ ID NO: 16 - protein, 151 aa, Artificial Sequence
  Description of Artificial Sequence Synthetic polypeptide
  Synthetic fusion protein Yap1 TF1a SV40 VP16 domains
MPETKQKRTAQNRAAQRAFRERKERKMKELEKKVQSLESIQQQNEVEATFLRDQLITLVNELKKYGPKKKRKVAPPTDVSLGDELHLDGEDVAMAHADALDDFDLDMLGDGDSPGPGFTPHDSAPYGALDMADFEFEQMFTDALGIDEYGG

SEQ ID NO: 17 - protein, 177 aa, Artificial Sequence
  Description of Artificial Sequence Synthetic polypeptide
  Synthetic fusion protein Yap1 TF1b SV40 VP16 domains
MKQDLDPETKQKRTAQNRAAQRAFRERKERKMKELEKKVQSLESIQQQNEVEATFLRDQLITLVNELKKYRPETRNDSKVLEYLARRDPNLGPKKKRKVAPPTDVSLGDELHLDGEDVAMAHADALDDFDLDMLGDGDSPGPGFTPHDSAPYGALDMADFEFEQMFTDALGIDEYGG

SEQ ID NO: 18 - protein, 782 aa, Artificial Sequence
  Description of Artificial Sequence Synthetic polypeptide
  Synthetic fusion protein Yap1 SV40 C1 domains
MSVSTAKRSLDVVSPGSLAEFEGSKSRHDEIENEHRRTGTRDGEDSEQPKKKGSKTSKKQDLDPETKQKRTAQNRAAQRAFRERKERKMKELEKKVQSLESIQQQNEVEATFLRDQLITLVNELKKYRPETRNDSKVLEYLARRDPNLHFSKNNVNHSNSEPIDTPNDDIQENVKQKMNFTFQYPLDNDNDNDNSKNVGKQLPSPNDPSHSAPMPINQTQKKLSDATDSSSATLDSLSNSNDVLNNTPNSSTSMDWLDNVIYTNRFVSGDDGSNSKTKNLDSNMFSNDFNFENQFDEQVSEFCSKMNQVCGTRQCPIPKKPISALDKEVFASSSILSSNSPALTNTWESHSNITDNTPANVIATDATKYENSFSGFGRLGFDMSANHYVVNDNSTGSTDSTGSTGNKNKKNNNNSDDVLPFISESPFDMNQVTNFFSPGSTGIGNNAASNTNPSLLQSSKEDIPFINANLAFPDDNSTNIQLQPFSESQSQNKFDYDMFFRDSSKEGNNLFGEFLEDDDDDKKAANMSDDESSLIKNQLINEEPELPKQYLQSVPGNESEISQKNGSSLQNADKINNGNDNDNDNDVVPSKEGSLLRCSEIWDRITTHPKYSDIDVDGLCSELMAKAKCSERGVVINAEDVQLALNKHMNGPKKKRKVCETGQNSAAHRADPDSAGTTTTSAAAVWAPKAVRCTGGLFFFHRDTTPAHAGETATPMAGGGGGGGGEAGSSDDCSSAASVSLRVGSHDEPCFSGDGDGDWMDDVRALASFLESDEDWLRCQTA

SEQ ID NO: 19 - protein, 197 aa, Artificial Sequence
  Description of Artificial Sequence Synthetic polypeptide
  Synthetic fusion protein Yap1 TF1a SV40 C1 domains
MPETKQKRTAQNRAAQRAFRERKERKMKELEKKVQSLESIQQQNEVEATFLRDQLITLVNELKKYGPKKKRKVCETGQNSAAHRADPDSAGTTTTSAAAVWAPKAVRCTGGLFFFHRDTTPAHAGETATPMAGGGGGGGGEAGSSDDCSSAASVSLRVGSHDEPCFSGDGDGDWMDDVRALASFLESDEDWLRCQTA

SEQ ID NO: 20 - protein, 223 aa, Artificial Sequence
  Description of Artificial Sequence Synthetic polypeptide
  Synthetic fusion protein Yap1 TF1b SV40 C1 domains
MKQDLDPETKQKRTAQNRAAQRAFRERKERKMKELEKKVQSLESIQQQNEVEATFLRDQLITLVNELKKYRPETRNDSKVLEYLARRDPNLGPKKKRKVCETGQNSAAHRADPDSAGTTTTSAAAVWAPKAVRCTGGLFFFHRDTTPAHAGETATPMAGGGGGGGGEAGSSDDCSSAASVSLRVGSHDEPCFSGDGDGDWMDDVRALASFLESDEDWLRCQTA

SEQ ID NO: 21 - protein, 596 aa, Artificial Sequence
  Description of Artificial Sequence Synthetic polypeptide
  Synthetic fusion protein Gat1 SV40 VP16 domains
MHVFFPLLFRPSPVLFIACAYIYIDIYIHCTRCTVVNITMSTNRVPNLDPDLNLNKEIWDLYSSAQKILPDSNRILNLSWRLHNRTSFHRINRIMQHSNSIMDFSASPFASGVNAAGPGNNDLDDTDTDNQQFFLSDMNLNGSSVFENVFDDDDDDDDVETHSIVHSDLLNDMDSASQRASHNASGFPNFLDTSCSSSFDDHFIFTNNLPFLNNNSINNNHSHNSSHNNNSPSIANNTNANTNTNTSASTNTNSPLLRRNPSPSIVKPGSRRNSSVRKKKPALKKIKSSTSVQSSATPPSNTSSNPDIKCSNCTTSTTPLWRKDPKGLPLCNACGLFLKLHGVTRPLSLKTDIIKKRQRSSTKINNNITPPPSSSLNPGAAGKKKNYTASVAASKRKNSLNIVAPLKSQDIPIPKIASPSIPQYLRSNTRHHLSSSVPIEAETFSSFRPDMNMTMNMNLHNASTSSFNNEAFWKPLDSAIDHHSGDTNPNSNMNTTPNGNLSLDWLNLNLGPKKKRKVAPPTDVSLGDELHLDGEDVAMAHADALDDFDLDMLGDGDSPGPGFTPHDSAPYGALDMADFEFEQMFTDALGIDEYGG

SEQ ID NO: 22 - protein, 141 aa, Artificial Sequence
  Description of Artificial Sequence Synthetic polypeptide
  Synthetic fusion protein Gat1 TF1a SV40 VP16 domains
MSNPDIKCSNCTTSTTPLWRKDPKGLPLCNACGLFLKLHGVTRPLSLKTDIIKKRGPKKKRKVAPPTDVSLGDELHLDGEDVAMAHADALDDFDLDMLGDGDSPGPGFTPHDSAPYGALDMADFEFEQMFTDALGIDEYGG

SEQ ID NO: 23 - protein, 152 aa, Artificial Sequence
  Description of Artificial Sequence Synthetic polypeptide
  Synthetic fusion protein Gat1 TF1b SV40 VP16 domains
MPSNTSSNPDIKCSNCTTSTTPLWRKDPKGLPLCNACGLFLKLHGVTRPLSLKTDIIKKRQRSSTKGPKKKRKVAPPTDVSLGDELHLDGEDVAMAHADALDDFDLDMLGDGDSPGPGFTPHDSAPYGALDMADFEFEQMFTDALGIDEYGG

SEQ ID NO: 24 - protein, 642 aa, Artificial Sequence
  Description of Artificial Sequence Synthetic polypeptide
  Synthetic fusion protein Gat1 SV40 C1 domains
MHVFFPLLFRPSPVLFIACAYIYIDIYIHCTRCTVVNITMSTNRVPNLDPDLNLNKEIWDLYSSAQKILPDSNRILNLSWRLHNRTSFHRINRIMQHSNSIMDFSASPFASGVNAAGPGNNDLDDTDTDNQQFFLSDMNLNGSSVFENVFDDDDDDDDVETHSIVHSDLLNDMDSASQRASHNASGFPNFLDTSCSSSFDDHFIFTNNLPFLNNNSINNNHSHNSSHNNNSPSIANNTNANTNTNTSASTNTNSPLLRRNPSPSIVKPGSRRNSSVRKKKPALKKIKSSTSVQSSATPPSNTSSNPDIKCSNCTTSTTPLWRKDPKGLPLCNACGLFLKLHGVTRPLSLKTDIIKKRQRSSTKINNNITPPPSSSLNPGAAGKKKNYTASVAASKRKNSLNIVAPLKSQDIPIPKIASPSIPQYLRSNTRHHLSSSVPIEAETFSSFRPDMNMTMNMNLHNASTSSFNNEAFWKPLDSAIDHHSGDTNPNSNMNTTPNGNLSLDWLNLNLGPKKKRKVCETGQNSAAHRADPDSAGTTTTSAAAVWAPKAVRCTGGLFFFHRDTTPAHAGETATPMAGGGGGGGGEAGSSDDCSSAASVSLRVGSHDEPCFSGDGDGDWMDDVRALASFLESDEDWLRCQTA

SEQ ID NO: 25 - protein, 187 aa, Artificial Sequence
  Description of Artificial Sequence Synthetic polypeptide
  Synthetic fusion protein Gat1 TF1a SV40 C1 domains
MSNPDIKCSNCTTSTTPLWRKDPKGLPLCNACGLFLKLHGVTRPLSLKTDIIKKRGPKKKRKVCETGQNSAAHRADPDSAGTTTTSAAAVWAPKAVRCTGGLFFFHRDTTPAHAGETATPMAGGGGGGGGEAGSSDDCSSAASVSLRVGSHDEPCFSGDGDGDWMDDVRALASFLESDEDWLRCQTA

SEQ ID NO: 26 - protein, 198 aa, Artificial Sequence
  Description of Artificial Sequence Synthetic polypeptide
  Synthetic fusion protein Gat1 TF1b SV40 C1 domains
MPSNTSSNPDIKCSNCTTSTTPLWRKDPKGLPLCNACGLFLKLHGVTRPLSLKTDIIKKRQRSSTKGPKKKRKVCETGQNSAAHRADPDSAGTTTTSAAAVWAPKAVRCTGGLFFFHRDTTPAHAGETATPMAGGGGGGGGEAGSSDDCSSAASVSLRVGSHDEPCFSGDGDGDWMDDVRALASFLESDEDWLRCQTA

SEQ ID NO: 27 - protein, 286 aa, Saccharomyces cerevisiae
MSDIEEGTPTNNGQQKERRKIEIKFIENKTRRHVTFSKRKHGIMKKAFELSVLTGTQVLLLVVSETGLVYTFSTPKFEPIVTQQEGRNLIQACLNAPDDEEEDEEEDGDDDDDDDDDGNDMQRQQPQQQQPQQQQQVLNAHANSLGHLNQDQVPAGALKQEVKSQLLGGANPNQNSMIQQQQHHTQNSQPQQQQQQQPQQQMSQQQMSQHPRPQQGIPHPQQSQPQQQQQQQQQLQQQQQQQQQQPLTGIHQPHQQAFANAASPYLNAEQNAAYQQYFQEPQQGQY

SEQ ID NO: 28 - protein, 126 aa, Saccharomyces cerevisiae
MDDICSMAENINRTLFNILGTEIDEINLNTNNLYNFIMESNLTKVEQHTLHKNISNNRLEIYHHIKKEKSPKGKSSISPQARAFLEQVFRRKQSLNSKEKEEVAKKCGITPLQVRVWFINKRMRSK

SEQ ID NO: 29 - protein, 210 aa, Saccharomyces cerevisiae
MNKIPIKDLLNPQITDEFKSSILDINKKLFSICCNLPKLPESVTTEEEVELRDILGFLSRANKNRKISDEEKKLLQTTSQLTTTITVLLKEMRSIENDRSNYQLTQKNKSADGLVFNVVTQDMINKSTKPYRGHRFTKENVRILESWFAKNIENPYLDTKGLENLMKNTSLSRIQIKNWVSNRRRKEKTITIAPELADLLSGEPLAKKKE

SEQ ID NO: 30 - protein, 650 aa, Saccharomyces cerevisiae
MSVSTAKRSLDVVSPGSLAEFEGSKSRHDEIENEHRRTGTRDGEDSEQPKKKGSKTSKKQDLDPETKQKRTAQNRAAQRAFRERKERKMKELEKKVQSLESIQQQNEVEATFLRDQLITLVNELKKYRPETRNDSKVLEYLARRDPNLHFSKNNVNHSNSEPIDTPNDDIQENVKQKMNFTFQYPLDNDNDNDNSKNVGKQLPSPNDPSHSAPMPINQTQKKLSDATDSSSATLDSLSNSNDVLNNTPNSSTSMDWLDNVIYTNRFVSGDDGSNSKTKNLDSNMFSNDFNFENQFDEQVSEFCSKMNQVCGTRQCPIPKKPISALDKEVFASSSILSSNSPALTNTWESHSNITDNTPANVIATDATKYENSFSGFGRLGFDMSANHYVVNDNSTGSTDSTGSTGNKNKKNNNNSDDVLPFISESPFDMNQVTNFFSPGSTGIGNNAASNTNPSLLQSSKEDIPFINANLAFPDDNSTNIQLQPFSESQSQNKFDYDMFFRDSSKEGNNLFGEFLEDDDDDKKAANMSDDESSLIKNQLINEEPELPKQYLQSVPGNESEISQKNGSSLQNADKINNGNDNDNDNDVVPSKEGSLLRCSEIWDRITTHPKYSDIDVDGLCSELMAKAKCSERGVVINAEDVQLALNKHMN

SEQ ID NO: 31 - protein, 510 aa, Saccharomyces cerevisiae
MHVFFPLLFRPSPVLFIACAYIYIDIYIHCTRCTVVNITMSTNRVPNLDPDLNLNKEIWDLYSSAQKILPDSNRILNLSWRLHNRTSFHRINRIMQHSNSIMDFSASPFASGVNAAGPGNNDLDDTDTDNQQFFLSDMNLNGSSVFENVFDDDDDDDDVETHSIVHSDLLNDMDSASQRASHNASGFPNFLDTSCSSSFDDHFIFTNNLPFLNNNSINNNHSHNSSHNNNSPSIANNTNANTNTNTSASTNTNSPLLRRNPSPSIVKPGSRRNSSVRKKKPALKKIKSSTSVQSSATPPSNTSSNPDIKCSNCTTSTTPLWRKDPKGLPLCNACGLFLKLHGVTRPLSLKTDIIKKRQRSSTKINNNITPPPSSSLNPGAAGKKKNYTASVAASKRKNSLNIVAPLKSQDIPIPKIASPSIPQYLRSNTRHHLSSSVPIEAETFSSFRPDMNMTMNMNLHNASTSSFNNEAFWKPLDSAIDHHSGDTNPNSNMNTTPNGNLSLDWLNLNL

SEQ ID NO: 32 - protein, 146 aa, Saccharomyces cerevisiae
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTV

SEQ ID NO: 33 - protein, 64 aa, Saccharomyces cerevisiae
PETKQKRTAQNRAAQRAFRERKERKMKELEKKVQSLESIQQQNEVEATFLRDQLITLVNELKKY

SEQ ID NO: 34 - protein, 90 aa, Saccharomyces cerevisiae
KQDLDPETKQKRTAQNRAAQRAFRERKERKMKELEKKVQSLESIQQQNEVEATFLRDQLITLVNELKKYRPETRNDSKVLEYLARRDPNL

SEQ ID NO: 35 - protein, 54 aa, Saccharomyces cerevisiae
IFTNNLPFLNNNSINNNHSHNSSHNNNSPSIANNTNANTNTNTSASTNTNSPLL

SEQ ID NO: 36 - protein, 64 aa, Saccharomyces cerevisiae
DDHFIFTNNLPFLNNNSINNNHSHNSSHNNNSPSIANNTNANTNTNTSASTNTNSPLLRRNPSP

SEQ ID NO: 37 - protein, 61 aa, Saccharomyces cerevisiae
KKEKSPKGKSSISPQARAFLEQVFRRKQSLNSKEKEEVAKKCGITPLQVRVWFINKRMRSK

SEQ ID NO: 38 - protein, 68 aa, Saccharomyces cerevisiae
STKPYRGHRFTKENVRILESWFAKNIENPYLDTKGLENLMKNTSLSRIQIKNWVSNRRRKEKTITIAP

SEQ ID NO: 39 - protein, 55 aa, Saccharomyces cerevisiae
RRKIEIKFIENKTRRHVTFSKRKHGIMKKAFELSVLTGTQVLLLVVSETGLVYTF

SEQ ID NO: 40 - protein, 81 aa, Saccharomyces cerevisiae
KERRKIEIKFIENKTRRHVTFSKRKHGIMKKAFELSVLTGTQVLLLVVSETGLVYTFSTPKFEPIVTQQEGRNLIQACLNA

SEQ ID NO: 41 - protein, 8 aa, Saccharomyces cerevisiae
  MOD_RES (2)..(3): Xaa can be any naturally occurring amino acid
  MOD_RES (6)..(6): Xaa can be any naturally occurring amino acid
GXXIRXRF

SEQ ID NO: 42 - protein, 8 aa, Saccharomyces cerevisiae
  MOD_RES (6)..(6): Xaa can be any naturally occurring amino acid
GGSIRXRF

SEQ ID NO: 43 - protein, 10 aa, Saccharomyces cerevisiae
GNSIRHRFRV

SEQ ID NO: 44 - protein, 78 aa, Herpes simplex virus 7
APPTDVSLGDELHLDGEDVAMAHADALDDFDLDMLGDGDSPGPGFTPHDSAPYGALDMADFEFEQMFTDALGIDEYGG

SEQ ID NO: 45 - protein, 124 aa, Zea mays
CETGQNSAAHRADPDSAGTTTTSAAAVWAPKAVRCTGGLFFFHRDTTPAHAGETATPMAGGGGGGGGEAGSSDDCSSAASVSLRVGSHDEPCFSGDGDGDWMDDVRALASFLESDEDWLRCQTA

SEQ ID NO: 46 - protein, 39 aa, Saccharomyces cerevisiae
  MOD_RES (4)..(4): Xaa can be any naturally occurring amino acid
  MOD_RES (10)..(10): Xaa can be any naturally occurring amino acid
  MOD_RES (12)..(12): Xaa can be any naturally occurring amino acid
  MOD_RES (15)..(15): Xaa can be any naturally occurring amino acid
  MOD_RES (17)..(17): Xaa can be any naturally occurring amino acid
  MOD_RES (19)..(20): Xaa can be any naturally occurring amino acid
  MOD_RES (24)..(26): Xaa can be any naturally occurring amino acid
  MOD_RES (28)..(30): Xaa can be any naturally occurring amino acid
  MOD_RES (34)..(35): Xaa can be any naturally occurring amino acid
IPEXELLDEXTXNFXSXLXXDLSXXXNXXXFEYXXEIAE

SEQ ID NO: 47 - protein, 42 aa, Saccharomyces cerevisiae
  MOD_RES (3)..(3): Xaa can be any naturally occurring amino acid
  MOD_RES (20)..(20): Xaa can be any naturally occurring amino acid
  MOD_RES (27)..(27): Xaa can be any naturally occurring amino acid
  MOD_RES (31)..(32): Xaa can be any naturally occurring amino acid
  MOD_RES (37)..(38): Xaa can be any naturally occurring amino acid
SYXIPENELLDEDTMNFISXLKNDLSXISNXXPFEYXXEIAE

SEQ ID NO: 48 - protein, 126 aa, Saccharomyces cerevisiae
SYAIPENELLDEDTMNFISSLKNDLSNISNSLPFEYPHEIAESYPIPENELLDEETMNFISNLKNDLSKLENNLPFEYSPEIAEQFSIPEGELLDEETLNFLSGLRRDLSRIENGMAFEYPQEIAE

SEQ ID NO: 49 - protein, 12 aa, Arabidopsis thaliana
LDLDLELRLGFA

SEQ ID NO: 50 - protein, 12 aa, Arabidopsis thaliana
IDLDLNLAPPME

SEQ ID NO: 51 - protein, 12 aa, Arabidopsis thaliana
QDLDLELRLGFA

SEQ ID NO: 52 - protein, 5 aa, Arabidopsis thaliana
LDLNL

SEQ ID NO: 53 - protein, 7 aa, Arabidopsis thaliana
  MOD_RES (6)..(6): Xaa can be any naturally occurring amino acid
LDLNLXP

SEQ ID NO: 54 - protein, 5 aa, Arabidopsis thaliana
  MOD_RES (2)..(2): Xaa can be any naturally occurring amino acid
  MOD_RES (4)..(4): Xaa can be any naturally occurring amino acid
LXLXL

SEQ ID NO: 55 - protein, 6 aa, Arabidopsis thaliana
  MOD_RES (4)..(5): Xaa can be any naturally occurring amino acid
DLNXXP

SEQ ID NO: 56 - protein, 5 aa, Arabidopsis thaliana
RLFGV

SEQ ID NO: 57 - protein, 8 aa, Arabidopsis thaliana
  MOD_RES (7)..(7): Xaa can be any naturally occurring amino acid
LRLFGVXL

SEQ ID NO: 58 - protein, 15 aa, Arabidopsis thaliana
GNSKTLRLFGVNMEC

SEQ ID NO: 59 - protein, 15 aa, Arabidopsis thaliana
GSSRTVRLFGVNLEC

SEQ ID NO: 60 - protein, 15 aa, Arabidopsis thaliana
GSSRTVRLFGVNLEC

SEQ ID NO: 61 - protein, 15 aa, Arabidopsis thaliana
GSSRTVRLFGVNLEC

SEQ ID NO: 62 - protein, 15 aa, Arabidopsis thaliana
TAGKRLRLFGVNMEC

SEQ ID NO: 63 - protein, 15 aa, Arabidopsis thaliana
RGEKRLRLFGVDMEC

SEQ ID NO: 64 - protein, 15 aa, Arabidopsis thaliana
STTKKLRLFGVDVEE

SEQ ID NO: 65 - protein, 15 aa, Arabidopsis thaliana
DAGRVLRLFGVNISP

SEQ ID NO: 66 - protein, 15 aa, Arabidopsis thaliana
PVQVVVRLFGVDIFN

SEQ ID NO: 67 - protein, 15 aa, Arabidopsis thaliana
ETGRVMRLFGVDISL

SEQ ID NO: 68 - protein, 15 aa, Arabidopsis thaliana
PVQTVVRLFGVNIFN

SEQ ID NO: 69 - protein, 15 aa, Arabidopsis thaliana
KAVTNFRLFGVSLAI

SEQ ID NO: 70 - protein, 15 aa, Arabidopsis thaliana
KTGTNFRLFGVTLDT

SEQ ID NO: 71 - protein, 15 aa, Arabidopsis thaliana
KTGTNFRLFGVSLVT

SEQ ID NO: 72 - protein, 15 aa, Arabidopsis thaliana
KAGTNFRLFGVTLDT

SEQ ID NO: 73 - protein, 15 aa, Arabidopsis thaliana
KAGTNFRLFGVSLAT

SEQ ID NO: 74 - protein, 15 aa, Arabidopsis thaliana
NAVASFRLFGVSLAT

SEQ ID NO: 75 - protein, 15 aa, Arabidopsis thaliana
GVGEGLKLFGVWLKG

SEQ ID NO: 76 - protein, 15 aa, Arabidopsis thaliana
EEEASPRLFGVPIGL

SEQ ID NO: 77 - protein, 15 aa, Arabidopsis thaliana
GEDLTPRLFGVSIGV

SEQ ID NO: 78 - protein, 14 aa, Arabidopsis thaliana
EEDEGLKLFGVKLE

SEQ ID NO: 79 - protein, 15 aa, Arabidopsis thaliana
SNMRKTKLFGVSLPS

SEQ ID NO: 80 - protein, 15 aa, Arabidopsis thaliana
GSSSAVKLFGVRLTD

SEQ ID NO: 81 - protein, 15 aa, Arabidopsis thaliana
CPNRGVKLFGVRLTE

SEQ ID NO: 82 - protein, 15 aa, Arabidopsis thaliana
YQTRVVRLFGVHLDT

SEQ ID NO: 83 - protein, 15 aa, Arabidopsis thaliana
VNKASVKLFGVNISS

SEQ ID NO: 84 - protein, 14 aa, Arabidopsis thaliana
ASLTKGKLFGVDLM

SEQ ID NO: 85 - protein, 14 aa, Arabidopsis thaliana
QSLSKARLFGVDLN

SEQ ID NO: 86 - protein, 14 aa, Arabidopsis thaliana
RLAAYAKLFGVPFE

SEQ ID NO: 87 - protein, 4 aa, Simian virus 40
  MOD_RES (3)..(3): Xaa can be any naturally occurring amino acid
KKXK

SEQ ID NO: 88 - protein, 7 aa, Simian virus 40
PKKKRKV

SEQ ID NO: 89 - protein, 9 aa, Homo sapiens
PAAKRVKLD

SEQ ID NO: 90 - protein, 9 aa, Escherichia coli
KLKIKRPVK

SEQ ID NO: 91 - protein, 16 aa, Xenopus laevis
  MOD_RES (3)..(12): Xaa can be any naturally occurring amino acid
KRXXXXXXXXXXKKKK

SEQ ID NO: 92 - protein, 16 aa, Xenopus laevis
KRPAATKKAGQAKKKK

SEQ ID NO: 93 - protein, 20 aa, Xenopus laevis
AVKRPAATKKAGQAKKKKLD

SEQ ID NO: 94 - protein, 25 aa, Caenorhabditis elegans
MSRRRKANPTKLSENAKKLAKEVEN

SEQ ID NO: 95 - protein, 8 aa, Homo sapiens
YNDFGNYN

SEQ ID NO: 96 - protein, 5 aa, Homo sapiens
FGNYN

SEQ ID NO: 97 - protein, 7 aa, Homo sapiens
SNFGPMK

SEQ ID NO: 98 - protein, 4 aa, Homo sapiens
NFGG

SEQ ID NO: 99 - protein, 6 aa, Homo sapiens
GPYGGG

SEQ ID NO: 100 - protein, 42 aa, Homo sapiens
GNYNNQSSNFGPMKGGNFGGRSSGPYGGGGQYFAKPRNQGGY

SEQ ID NO: 101 - protein, 45 aa, Homo sapiens
FGNYNQQPSNYGPMKSGNFGGSRNMGGPYGGGNYGPGGSGGSGGY

SEQ ID NO: 102 - protein, 43 aa, Homo sapiens
FGNYNSQSSSNFGPMKGGNYGGRNSGPYGGGYGGGSASSSSGY

SEQ ID NO: 103 - protein, 49 aa, Xenopus laevis
FGNYNQQSSNYGPMKSGGNFGGNRSMGGGPYGGGNYGPGNASGGNGGGY

SEQ ID NO: 104 - protein, 5 aa, Saccharomyces cerevisiae
KIPIK